(19) United States
(12) Patent Application Publication
(10) Pub. No.: US 2024/0386376 A1
(43) Pub. Date: Nov. 21, 2024

(54) SYSTEMS AND METHODS FOR GENERATING LONGITUDINAL DATA PROFILES FROM MULTIPLE DATA SOURCES

(71) Applicant: AbbVie Inc., North Chicago, IL (US)

(72) Inventors: Pankaj Dubey, Lake Villa, IL (US); Sanjeev Garr, Volo, IL (US); Jessica Yassick, Crystal Lake, IL (US); Shannon Sword, Deerfield, IL (US); Anandbabu Padmanaban, Waukegan, IL (US); Denise Campbell, Lake Forest, IL (US)

(21) Appl. No.: 18/666,929

(22) Filed: May 17, 2024

Related U.S. Application Data
(63) Continuation of application No. 15/087,438, filed on Mar. 31, 2016, now abandoned.
(60) Provisional application No. 62/141,590, filed on Apr. 1, 2015.
(60) Provisional application No. 62/195,160, filed on Jul. 21, 2015.

(51) Int. Cl. G06Q 10/10; G06F 16/21; G06F 16/2457; G06F 16/81; G06F 21/60; G06F 21/62; G16H 10/60

(52) U.S. Cl. CPC G06Q 10/10; G06F 16/21; G06F 16/24573; G06F 16/81; G06F 21/602; G06F 21/6245; G16H 10/60; G06Q 2220/10

(57) ABSTRACT

A computer-implemented method for generating a longitudinal data profile from multiple disparate data sources is provided. The method includes storing, at a central data hub, first de-identified data received from a first data source, the first de-identified data including a plurality of data records having encrypted identifying data and an anonymous ID assigned to each record, wherein the anonymous ID is assigned based on a master list that includes a list of identifiers and corresponding anonymous IDs for each identifier. The method further includes storing second de-identified data received from a second data source, and storing third de-identified data received from a third data source. The method further includes processing the first, second, and third de-identified data to link the first, second, and third de-identified data using the anonymous ID, and generating the longitudinal data profile from the linked first, second, and third de-identified data.

### CROSS REFERENCE TO RELATED APPLICATIONS

This application is a continuation of U.S. Non-Provisional patent application Ser. No. 15/087,438, filed on Mar. 31, 2016, which claims priority to U.S. Provisional Patent Application No. 62/141,590, filed on Apr. 1, 2015, and U.S. Provisional Patent Application No. 62/195,160, filed on Jul. 21, 2015, the entire disclosures of which are hereby incorporated by reference in their entirety.

### FIELD OF USE

The disclosed subject matter relates to data processing between multiple computers in a digital data processing system and, more particularly, to generating longitudinal data profiles from a plurality of disparate data sources.

### BACKGROUND

In digital data processing systems, vast quantities of data may be stored in a large number of separate databases. Such digital data processing systems are prevalent in, for example, the financial industry, the healthcare industry, the automotive industry, the insurance industry etc. In these industries, data stored in different databases may have still common parameters (e.g., data associated with a specific entity, time, date, location, person, etc. may be stored in multiple databases).

However, data stored in different databases may be stored in different formats or encrypted using different encryption algorithms. Further, some data may be encrypted while other data remains unencrypted. Accordingly, when attempting to consolidate all data associated with a common parameter (e.g., to perform analytics on the data), it may be challenging to retrieve and combine all available data to generate a single, comprehensive record associated with the common parameter.

For example, in the healthcare industry, when a patient is consulting with a healthcare provider (HCP), such as a doctor or a nurse practitioner, the HCP may prescribe a specific healthcare product or service to the patient (e.g., as a part of the patient's diagnosis or treatment). As an example, the HCP may prescribe a medication (e.g., a pharmaceutical or biologic product), a medical device (e.g., an oxygen cart), a surgical procedure (e.g., installation of a pacemaker), or a combination of any of these items. As another example, the healthcare provider may refer the patient to another healthcare provider who is a specialist in a specific field (e.g., a general practice doctor may refer the patient to a cardiologist, or a rheumatologist). The patient may receive little training or information regarding their medication or treatment regimen other than the standard safety or usage information associated with the medication.

As part of the prescription process, data related to the patient and the prescription(s) may be generated at a plurality of disparate data sources. Data may be independently generated at a healthcare provider (HCP) database, a pharmacy database, an insurance company database, a claims database, a patient care model database, etc. The patient may also participate in one or more patient support programs associated with the prescription product. These patient support programs may have their own databases that generate and store additional data. Moreover, over time, the patient may visit multiple HCPs, fill prescriptions at multiple pharmacies, and/or have coverage with multiple insurance companies. Each HCP, pharmacy, and insurance company typically has its own database and independently generates data for storage in that database.

Notably, these disparate data sources are not generally in contact with one another. Further, the data at each data source may be stored in different formats or schemas. Moreover, first data from a first data source may be de-identified data, which includes anonymized data that does not identify the particular patient for legal or other reasons, and second data from a second data source may be identified data, which includes non-anonymized data that does identify the particular patient. For example, under national and/or regional patient privacy regulations such as the United States Health Insurance Portability and Accountability Act of 1996, only certain covered entities may be authorized to view identified patient protected health information (PHI) data. Matching or linking these two types of data sources (e.g., de-identified and identified, or de-identified from two different sources) can be challenging.

Further, in the healthcare industry, a party (e.g., a pharmaceutical company, an insurance company, a HCP, a pharmacy, etc.) may wish to analyze healthcare data to identify trends or generate statistics. For example, a party may want to analyze the effectiveness of a patient support program associated with a particular prescription product. In at least some known healthcare data systems, the party would need to attempt to gather and consolidate data from a plurality of disparate data sources, which may be costly, time-consuming, and may result in delays in a patient receiving optimal care. Further, data from different data sources may be stored in different formats or schemas, and de-identified data may be irreconcilable with identified data. Accordingly, in the absence of a comprehensive longitudinal data profile that provides a substantially complete record regarding a patient and at least one prescription product, in at least some known healthcare data systems, effective data analysis may be elusive and difficult to achieve.

### BRIEF DESCRIPTION OF THE DISCLOSURE

In one aspect, a computer-implemented method for generating a longitudinal data profile from multiple disparate data sources is provided. The method includes storing, at a central data hub, first de-identified data received from a first data source, the first de-identified data including a plurality of data records having encrypted identifying data and an anonymous ID assigned to each record, wherein the anonymous ID is assigned based on a master list that includes a list of identifiers and corresponding anonymous IDs for each identifier. The method further includes storing, at the central data hub, second de-identified data received from a second data source, the second de-identified data including a plurality of data records having encrypted identifying data and an anonymous ID assigned to each record, wherein the anonymous ID is assigned based on the master list. The method further includes storing, at the central data hub, third de-identified data received from a third data source, the third de-identified data including a plurality of data records having encrypted identifying data and an anonymous ID assigned to each record, wherein the anonymous ID is assigned based on the master list. The method further includes processing, at the central data hub, the first, second, and third de-identified data to link the first, second, and third de-identified data using the anonymous ID, and generating, at the central data hub, the longitudinal data profile from the linked first, second, and third de-identified data by organizing the first, second, and third de-identified data to generate a comprehensive profile that includes data from multiple disparate data sources.

In another aspect, a computer-implemented method for generating a longitudinal data profile from multiple disparate data sources is provided. The method includes storing, at a central data hub, first de-identified data received from a claims data source, the first de-identified data including a plurality of data records having encrypted identifying data and an anonymous ID assigned to each record, wherein the claims data source stores insurance claims data associated with medical or pharmaceutical insurance coverage for patients, wherein the insurance claims data is encrypted at the claims data source using an encryption algorithm, and wherein the anonymous ID is assigned based on a patient master list that includes a list of patient identifiers and corresponding anonymous IDs for each patient identifier. The method further includes storing, at the central data hub, second de-identified data received from a services data source, the second de-identified data including a plurality of data records having encrypted identifying data and an anonymous ID assigned to each record, wherein the services data source stores services data that indicates whether patients are enrolled in a patient support program, wherein the services data is encrypted using the encryption algorithm, and wherein the anonymous ID is assigned based on the patient master list. The method further includes storing, at the central data hub, third de-identified data received from a pharmacy data source, the third de-identified data including a plurality of data records having encrypted identifying data and an anonymous ID assigned to each record, wherein the pharmacy data source stores pharmacy data associated with a prescription product for patients, wherein the pharmacy data is encrypted using the encryption algorithm, and wherein the anonymous ID is assigned based on the patient master list. The method further includes processing, at the central data hub, the first, second, and third de-identified data to link the first, second, and third de-identified data using the anonymous ID, and generating, at the central data hub, the longitudinal data profile for a patient from the linked first, second, and third de-identified data by organizing the first, second, and third de-identified data to generate a comprehensive history for the patient that includes data from multiple disparate healthcare data sources.

In another aspect, a central data hub for generating a longitudinal data profile from multiple disparate data sources is provided. The central data hub is configured to store first de-identified data received from a claims data source, the first de-identified data including a plurality of data records having encrypted identifying data and an anonymous ID assigned to each record, wherein the claims data source stores insurance claims data associated with medical or pharmaceutical insurance coverage for patients, wherein the insurance claims data is encrypted at the claims data source using an encryption algorithm, and wherein the anonymous ID is assigned based on a patient master list that includes a list of patient identifiers and corresponding anonymous IDs for each patient identifier. The central data hub is further configured to store second de-identified data received from a services data source, the second de-identified data including a plurality of data records having encrypted identifying data and an anonymous ID assigned to each record, wherein the services data source stores services data that indicates whether patients are enrolled in a patient support program, wherein the services data is encrypted using the encryption algorithm, and wherein the anonymous ID is assigned based on the patient master list. The central data hub is further configured to store third de-identified data received from a pharmacy data source, the third de-identified data including a plurality of data records having encrypted identifying data and an anonymous ID assigned to each record, wherein the pharmacy data source stores pharmacy data associated with a prescription product for patients, wherein the pharmacy data is encrypted using the encryption algorithm, and wherein the anonymous ID is assigned based on the patient master list. The central data hub is further configured to process the first, second, and third de-identified data to link the first, second, and third de-identified data using the anonymous ID, and generate the longitudinal data profile for a patient from the linked first, second, and third de-identified data by organizing the first, second, and third de-identified data to generate a comprehensive history for the patient that includes data from multiple disparate healthcare data sources.

In yet another aspect, a computer-implemented method for directly providing a user with an estimated patient cost for a prescription product and a co-pay eligibility determination for the prescription product is provided. The method includes receiving, at a host computing device, from a remote user computing device, a user request for the estimated patient cost and the co-pay eligibility determination, wherein the user request includes patient identification information, and wherein the remote user computing device and the host computing device are linked together via a wide area network that includes the Internet, retrieving, by the host computing device, from at least one database, benefits data and medication history data based on the patient identification information, generating, at the host computing device, the estimated patient cost and the co-pay eligibility determination based on the patient identification information and the retrieved benefits data and medication history data, and returning the estimated patient cost and the co-pay eligibility determination to the user.

### BRIEF DESCRIPTION OF THE DRAWINGS

FIGS. 1-49 show example embodiments of the methods and systems described herein.

FIG. 1 is a simplified block diagram of an example healthcare data processing system that includes a healthcare data analysis device and other computing devices in accordance with one example embodiment of the present disclosure.

FIG. 2 is an expanded block diagram of an example embodiment of a server architecture of the healthcare data processing system including the healthcare data analysis device and a plurality of other computing devices in accordance with one example embodiment of the present disclosure.

FIG. 3 illustrates an example configuration of a client system shown in FIGS. 1 and 2.

FIG. 4 illustrates an example configuration of a server system shown in FIGS. 1 and 2.

FIG. 5 illustrates an example architecture of a healthcare data processing system.

FIG. 6 illustrates an example data flow from a benefits verifier to a central data hub.

FIGS. 7A and 7B illustrate example data flows from a marketing database to a central data hub.

FIG. 8 illustrates an example data flow from a marketing database to a data analysis party.

FIG. 9 illustrates multiple examples of data flow from a pharmacy data source to a marketing database and a pharmacy database.

FIG. 10A illustrates an example of data flow in the system shown in FIG. 5.

FIG. 10B illustrates real-time data integration in the system shown in FIG. 5.

FIG. 11 illustrates a simplified overview of an example data flow in the system shown in FIG. 5.

FIG. 12 illustrates an example of an architecture of a central data hub.

FIG. 13 illustrates an example of an architecture of a central data hub.

FIG. 14 illustrates an example flow from a landing area to a staging area under a generic table ETL approach in the architecture shown in FIGS. 12 and 13.

FIG. 15 illustrates an example flow from a staging area to an APLD under a generic table ETL approach in the architecture shown in FIGS. 12 and 13.

FIG. 16 illustrates an example LPD claims conceptual data model for an APLD in the architecture shown in FIGS. 12 and 13.

FIG. 17 illustrates an example conceptual data model for patient support program data in the architecture shown in FIGS. 12 and 13.

FIG. 18 illustrates an example conceptual data model for CRM kit delivery data in the architecture shown in FIGS. 12 and 13.

FIG. 19 illustrates an example patient referral, benefits summary, prescription shipment, and patient calls conceptual data model for an APLD in the architecture shown in FIGS. 12 and 13.

FIG. 20 illustrates an example co-pay card redemption conceptual data model for an APLD in the architecture shown in FIGS. 12 and 13.

FIG. 21 illustrates an example data flow from a data consolidator to various components of the system shown in FIG. 5.

FIGS. 22A and 22B are an example flow diagram for calculating an estimated patient cost for a prescription product.

FIGS. 23A and 23B are pseudocode for an example algorithm for calculating an estimated patient cost for a prescription product.

FIGS. 24-49 include screenshots of one example of a patient application as part of a coordinated care platform, as shown in FIG. 5.

Like numbers in the Figures indicate the same or functionally similar components.

### DETAILED DESCRIPTION OF THE DISCLOSURE

Embodiments of the methods and systems described herein enable a party to collect and organize data from multiple disparate data sources to generate data profiles including longitudinal data profiles. For example, when a patient visits and consults with a healthcare provider (HCP) (e.g., a doctor, a nurse practitioner, or the like), the HCP may prescribe/order a product (e.g., a prescription medication) and/or service (e.g., a treatment, a surgical procedure, a referral to a specialist). As used herein, the term prescription product may include drugs, pharmaceutical products, medical devices, medical therapies, surgical procedures, diagnostic procedures, as well as any other products or procedures that require a prescription from an HCP or generate a claim with an insurance company. The embodiments disclosed herein also provide a patient application that facilitates improving patient adherence and collecting information regarding patient adherence.

As part of the prescription process for a particular patient, data related to the patient and a prescription product prescribed to the patient may be generated at a plurality of disparate data sources. Data may be independently generated at a healthcare provider (HCP) database, a pharmacy database, an insurance company database, a claims database, and/or a health system database, etc. Data may also be generated and/or retrieved prior to a patient encountering a HCP. For example, data may be retrieved from users of an electronic healthcare information portal, social media, a patient application associated with a particular medication or treatment regimen, etc. (e.g., to evaluate and improve marketing and educational information on a prescription product). The patient may also participate in one or more services (e.g., patient support programs) associated with the prescription product. These patient support programs may have their own databases that generate and store additional data. Moreover, over time, the patient may visit multiple HCPs, fill prescriptions at multiple pharmacies, visit multiple hospitals, and/or have coverage with multiple insurance companies (e.g., through the same or different employers). Each HCP, pharmacy, hospital, and insurance company typically has its own database and independently generates data for storage in that database. Data from these disparate data sources may be processed to improve patient experiences with a prescription product at any time (e.g., prior to the patient meeting with an HCP and/or following use of the prescription product).

Notably, these disparate data sources are not generally in contact with one another. Further, the data at each data source may be stored in different formats or schemas. In some embodiments, first data from a first data source is de-identified data, which includes anonymized data that does not identify the particular patient for legal or other reasons, and second data from a second data source is identified data, which includes non-anonymized data that does identify the particular patient. For example, under the United States Health Insurance Portability and Accountability Act of 1996, only certain covered entities are authorized to view identified patient PHI data. These two types of patient data (e.g., de-identified and identified data, or de-identified data from two different data sources) can be difficult to match or link up.

The systems and methods described herein facilitate collecting and consolidating data from a plurality of disparate data sources at a central data hub to generate a plurality of longitudinal data profiles. As described herein, each longitudinal data profile includes healthcare data collected from one or more data sources for a particular patient. The data is (i) collected, (ii) encrypted to de-identify the particular patient, (iii) assigned an anonymous ID, and (iv) formatted into a single format so that it can be stored in the central data hub, wherein the formatting includes organizing the data into a predetermined structure such as organizing the data in chronological order. The longitudinal data profile may include healthcare data collected from a single data source for a single prescription-related event (e.g., one instance of fill confirmation data), or may include healthcare data collected from multiple data sources for one or more events.

The disparate data sources may include, but are not limited to, claims data sources, services, marketing, and customer relationship management (CRM) data sources (collectively referred to herein as “services data sources”), and pharmacy data sources. The data sources may also include other types of data sources (e.g., a patient application, electronic medical device data sources, drug interaction data sources, patient care applications including an instant benefit verification application and a physician portal for managing and tracking prescriptions, clinical data sources, electronic social media, search engines, electronic educational portals, and/or patient information data sources). Further, the data collected from the disparate data sources may include data both associated with a particular prescription product and data that is not associated with the prescription product.

Claims data sources are data sources that typically store, process and/or have access to insurance claims data associated with medical or pharmaceutical insurance coverage. Claims data sources may include, for example, a benefits verifier that provides a benefits summary in response to a benefits verification request, a co-pay data source, a prior authorization data source, an employer data source that provides work productivity data, and a clinical data source that provides electronic health record (EHR) data to the central data hub.

In the example embodiment, the claims data sources generally include an encryption module that encrypts data stored therein using an encryption algorithm provided by a syndicated data provider. Once the data is encrypted, the encrypted data is transmitted to the syndicated data provider. In the example embodiment, the syndicated data provider then uses an encrypted patient master to assign an anonymous ID to the encrypted data. In another embodiment, the claims data is sent to the syndicated data provider, a portion of which may be un-encrypted, and the syndicated data provider assigns the anonymous ID to the encrypted data using a patient master and de-identifies the claims data.

In the example embodiment, the patient master may include a roster having a list of patient identifiers and an anonymous ID associated with each patient identifier. The patient identifier may be patient information (e.g., name, address, etc.) in an encrypted form or an un-encrypted form. In the example embodiment, the syndicated data provider determines the patient identifier from the encrypted data, and assigns the appropriate anonymous ID if the patient identifier is already listed on the patient master. If the patient identifier is not already listed on the patient master (e.g., for new patients), the syndicated data provider may generate a new, unique anonymous ID.

Once the anonymous ID is assigned, the data may be referred to as “de-identified” data. Notably, the same encryption algorithm (e.g., the encryption algorithm of syndicated data provider) may be used by one or more of the claims data sources. Accordingly, first encrypted data for a patient (Patient A) that is received at the syndicated data provider from a first claims data source will be assigned the same anonymous ID as second encrypted data for the same patient (Patient A) that is received at the syndicated data provider from a second claims data source. The de-identified data is then transmitted to the central data hub.

Services data sources typically store, process and/or have access to data that generally relates to services (e.g., patient support programs) provided to a patient as part of the prescription process. For example, services data sources may include an adherence or compliance tracking data source that provides data indicating whether the patient has taken the prescription product. In one embodiment, the adherence tracking data may be received through a patient application. As explained further below, the adherence tracking data may be stored in a central data hub. Services data sources may further include a customer relationship management (CRM) data source that provides data from a CRM program that assists the patient during the prescription process, a healthcare coordinator data source that provides data related to a healthcare coordinator (e.g., virtual or live) that guides the patient through the prescription process, a call center data source, and a nursing data source. Services data sources may also provide symptom tracking data, patient related outcomes (PRO) assessment data, consumption data from tracking devices such as auto injectors, wearable injectors, infusion pumps, and injection syringes, work productivity and absenteeism data, clinical data (e.g., from electronic medical record (EMR)) data sources, and/or medication fill history data.

In the example embodiment, the services data sources transmit their data either directly or indirectly to a marketing database. Unlike the claims data sources, the services data sources and the marketing database may not include an encryption module. Accordingly, to encrypt the data from the services data sources, the marketing database transmits at least some of that data to a claims switch that includes an encryption module. In the example embodiment, a claims switch is a computing device associated with an entity that routes pharmaceutical and/or medical claims from a pharmacy to a payer (e.g., an insurance company). The claims switch encrypts the received data using an encryption module (with the encryption algorithm of the syndicated data provider) and transmits the encrypted data to the syndicated data provider. The syndicated data provider then assigns an anonymous ID to the encrypted data, and transmits the de-identified data to the central data hub. In another embodiment, the services data is encrypted at the services data sources and sent to the syndicated data provider.

Pharmacy data sources typically store data that is generally related to the filling of the prescription product. Data from the pharmacy data sources may include, for example, fill history data, prescription transfer data, benefit verification data, prior authorization data, insurance data, prescription product manufacturing information, shipment tracking data and data related to a grace filling of the prescription product. For example, data from the pharmacy data sources may include data related to record information (e.g., date and time a record was generated at the pharmacy data source, a pharmacy national provider identifier (NPI) number, etc.), patient preferences (e.g., if the patient prefers easy to open product containers, if the patient receives electronic notifications, etc.), patient demographics (e.g., patient referral source, date and time patient referral was received at the pharmacy data source, etc.), patient services (e.g., whether a patient was offered access to a support program, whether a patient is currently a member of the support program, etc.), status information (e.g., date when a prior authorization expires, date and time a patient was assigned a prior authorization status, etc.), HIPAA authorization (e.g., whether patient executed HIPAA authorization, date HIPAA authorization was discussed with patient, etc.), prescriber demographics (e.g., prescriber name, prescriber state license number, etc.), prescription/shipment information (e.g., date prescription was written, number of refills remaining, etc.), insurance information (e.g., name of pharmacy benefits manager, type of benefit coverage, etc.), claim information (e.g., type of coverage applied to claim, policy expiration date, etc.), transfer information (e.g., name of pharmacy receiving transfer, date of beginning of patient transfer, etc.), and other information (e.g., pertinent notes on patient or service delivery). The pharmacy data sources transmit data to an aggregated pharmacy database. Similar to the data from the claims data sources and the services data sources, this data is encrypted, assigned an anonymous ID, and transmitted as de-identified data to the central data hub. The data may be encrypted at the pharmacy data sources or at the syndicated data provider.

As described above, in the example embodiment, the central data hub receives and stores de-identified data that originated at the claims data sources, the services data sources, and the pharmacy data sources. The de-identified data may be available for processing for a pre-determined period of time. In some examples, all or portions of the data at the central data hub may have been de-identified using the encryption algorithm and assigned an anonymous ID from the encrypted patient master of the syndicated data source. Accordingly, although the data is de-identified, all data for a particular patient may have the same anonymous ID. For each patient, the central data hub generates a longitudinal data profile by organizing the de-identified data using the anonymous ID. For example, the longitudinal data profile may be generated by chronologically ordering all of the de-identified data for each patient. As the longitudinal data profile includes data for the patient that has been received from a plurality of disparate data sources, the longitudinal data profile provides a comprehensive, substantially complete history for the patient. Further, the longitudinal data profile includes significantly more information than could be obtained from a single data source.

Using a data analyzer computing device included in or in communication with the central data hub, longitudinal data profiles may be analyzed to determine useful information. In the example embodiment, the data analyzer is a specialized computing device for analyzing longitudinal data profiles generated as described herein, and includes one or more processors and a memory. The data analyzer may be configured to electronically receive one or more inputs from a user, and compare a plurality of longitudinal data profiles based on the one or more inputs. The data analyzer may output results of the comparison for review by the user. For example, an analyzer party (e.g., a pharmaceutical company, an insurance company, an HCP, a pharmacy, etc.) may wish to analyze healthcare data to identify one or more trends or generate statistics. For example, an analyzer party may want to analyze the effectiveness of a patient support program associated with a particular prescription product. In some embodiments, the patient support program may be provided by the party doing the analysis. As another example, an analyzer party or a patient may want to compare patient adherence information for multiple patients on the same treatment regimen. The patient adherence information may be collected, for example, by receiving patient adherence information through a patient application in which individual patients input their respective adherence information.

Using the data analyzer, the analyzer party can compare, for example, patients using the prescription product who participate in the patient support program against patients using the prescription product who do not participate in the patient support program. Further, the data analyzer may be used to compare, for a particular patient, a patient condition before participation in the patient support program against a patient condition during or after participation in the patient support program. In addition, the data analyzer may compare the adherence or compliance to a treatment regimen between multiple patients. As used herein, a “patient condition” may include, for example, a patient outcome and/or healthcare costs. The data analyzer may be used to compare longitudinal data profiles for one or more patients over the same time period and/or over different time periods to determine changes in patient data. Accordingly, based on the results output by the data analyzer, the analyzer party can determine a change in patient data (e.g., whether participation in the patient support program is generally associated with an improvement in patient condition over time or an increase in patient adherence). An improvement in patient condition may be based on, for example, a number of insurance claims made during a period of time that the patient participates in the patient support program, medical costs associated with the patient during a period of time that the patient participates in the patient support program, patient feedback, patient satisfaction, etc. To perform such an analysis, without the central data hub described herein, the analyzer party would need to attempt to gather and reconcile data from a plurality of disparate data sources, which may be costly and time-consuming. As described above, data from different data sources may be stored in different formats or schemas. Further, de-identified data may be irreconcilable with identified data.

However, the systems and methods described herein provide a central data hub that collects data from disparate data sources and generates a comprehensive longitudinal data profile for a patient. By generating longitudinal data profiles for one or more patients, the systems and methods described herein are further configured to analyze the longitudinal healthcare data. For example, to identify changes in patient condition, a first portion of a longitudinal data profile corresponding to a first time when the particular patient was not enrolled in a patient support program may be compared to a second portion of the longitudinal data profile corresponding to a second time when the particular patient was enrolled in the patient support program. In another example, when the central data hub includes longitudinal data profiles for both patients participating in a patient support program and patients not participating in a patient support program, using the data analyzer included in or in communication with the central data hub, those longitudinal profiles can be compared against each other to determine an efficacy of the patient support program (e.g., by determining whether patients participating in the patient support program have, on the whole, an improved condition relative to patients not participating in the patient support program). Accordingly, the longitudinal data profiles generated using the systems and methods described herein provide a powerful tool for healthcare data analytics and solve technical problems of existing healthcare data systems.

At least one of the technical problems addressed by the systems and methods described herein includes: (i) inability to consolidate data from multiple healthcare data sources; (ii) inability to reconcile de-identified data with identified data; (iii) lack of a comprehensive longitudinal data profile for a patient that includes data from a plurality of disparate data sources; and (iv) inability to analyze large quantities of data related to services associated with a prescription product. Certain embodiments described herein may address one or more of these technical problems in a prompt, relatively automated fashion.

The methods and systems described herein may be implemented using computer programming or engineering techniques including computer software, firmware, hardware, or any combination or subset thereof, wherein the technical effects may be achieved by performing at least one of the following steps: (a) storing first de-identified data received from a claims data source, the first de-identified data including first encrypted data and an anonymous ID; (b) storing second de-identified data received from a services data source, the second de-identified data including second encrypted data and the anonymous ID; (c) storing third de-identified data received from a pharmacy data source, the third de-identified data including third encrypted data and the anonymous ID; (d) processing the first, second, and third de-identified data to link the first, second, and third de-identified data using the anonymous ID; and (e) generating the longitudinal data profile from the linked first, second, and third de-identified data.

The resulting technical effect achieved by the systems and methods described herein may include at least one of: (i) collecting data from a plurality of disparate data sources at a central data hub; (ii) matching de-identified data with data that is initially identified data; (iii) generating comprehensive longitudinal data profiles for patients using data collected from a plurality of disparate data sources; and (iv) analyzing large quantities of healthcare data to determine the efficacy of one or more services associated with a prescription product in real time (e.g., relatively quickly and automatically) to generate a real world output.

The following detailed description illustrates embodiments of the disclosure by way of example and not by way of limitation. It is contemplated that the embodiments have general application to processing healthcare data in a variety of applications.

As used herein, the term “database” may refer to either a body of data, a relational database management system (RDBMS), or to both. As used herein, a database may include any collection of data including hierarchical databases, relational databases, flat file databases, object-relational databases, object-oriented databases, and any other structured collection of records or data that is stored in a computer system. The above examples are example only and thus are not intended to limit in any way the definition and/or meaning of the term database. Examples of RDBMS's include, but are not limited to including, Oracle® Database, MySQL, Teradata, IBM® DB2, Microsoft® SQL Server, Sybase®, and PostgreSQL. However, any database may be used that enables the systems and methods described herein. (Oracle is a registered trademark of Oracle Corporation, Redwood Shores, California; IBM is a registered trademark of International Business Machines Corporation, Armonk, New York; Microsoft is a registered trademark of Microsoft Corporation, Redmond, Washington; and Sybase is a registered trademark of Sybase, Dublin, California.)

In one embodiment, a computer program is provided, and the program is embodied on a computer-readable medium. In an example embodiment, the system is executed on a single computer system, without requiring a connection to a sever computer. In a further embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited Reading, Berkshire, United Kingdom). The application is flexible and designed to run in various different environments without compromising any major functionality. In some embodiments, the system includes multiple components distributed among a plurality of computing devices. One or more components may be in the form of computer-executable instructions embodied in a computer-readable medium.

As used herein, an element or step recited in the singular and preceded with the word “a” or “an” should be understood as not excluding plural elements or steps, unless such exclusion is explicitly recited. Furthermore, references to “example embodiment” or “one embodiment” of the present disclosure are not intended to be interpreted as excluding the existence of additional embodiments that also incorporate the recited features.

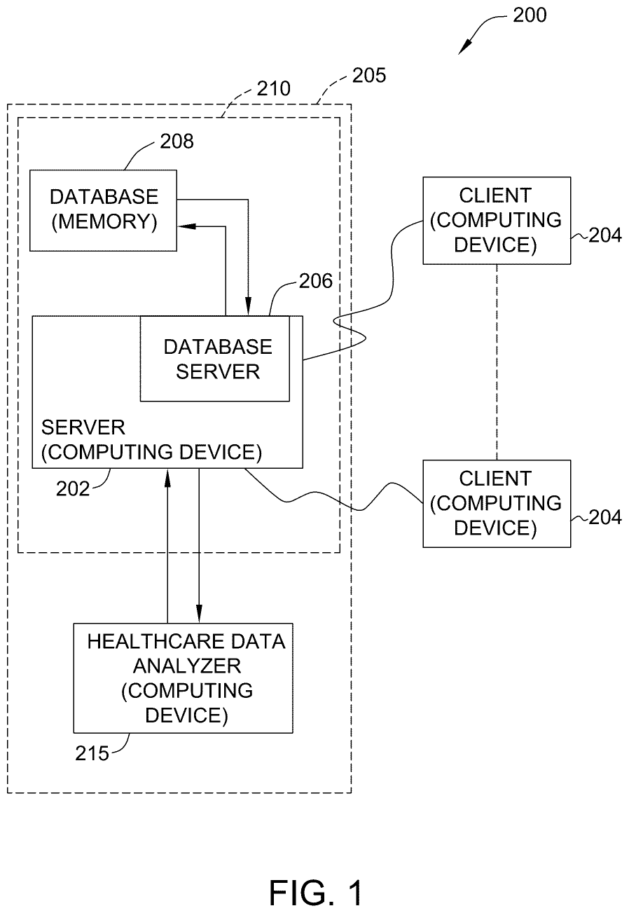

FIG. 1 is a simplified block diagram of one embodiment of a healthcare data processing system 200 that includes a data hub 205 having a data aggregator 210 and a data analyzer 215. In the example embodiment, data aggregator 210 includes a server system 202 including a database server 206, and a database 208 in communication with server system 202. Data analyzer 215 includes a processing device and a memory. System 200 further includes a plurality of client subsystems, also referred to as client systems 204 or client computing devices, connected to server system 202. In one embodiment, client systems 204 are computers including a web browser, such that server system 202 is accessible to client systems 204 using the Internet or another network. Client systems 204 are interconnected to the Internet or another network through many interfaces including a network, such as a local area network (LAN) and/or a wide area network (WAN), dial-in connections, cable modems, wireless-connections, and special high-speed ISDN lines. Client systems 204 may be any device capable of interconnecting to the Internet including a web-based phone, personal digital assistant (PDA), watch, medical device, kiosk, laptop computer, desktop computer, netbook, tablet, phablet, or other web-connectable equipment.

Database server 206 is connected to database 208 containing information on a variety of matters, as described below in greater detail. In one embodiment, database 208 is stored on server system 202 and may be accessed by potential users at one of client systems 204 by logging onto server system 202 through one of client systems 204. Database 208 is also accessible to healthcare data analyzer 215. In an alternative embodiment, database 208 is stored remotely from server system 202 and may be non-centralized (e.g., in a cloud computing configuration). Server system 202 could be any type of computing device configured to perform the steps described herein. Additionally, healthcare data analyzer 215 is in communication with server system 202. In some implementations, healthcare data analyzer 215 is incorporated into or integrated within server system 202. As described herein, server system 202 collects and stores data from a plurality of data sources (e.g., client systems 204) in database 208, and healthcare data analyzer 215 enables a user to view and analyze the healthcare data stored in database 208.

Database 208 and server system 202 (including database server 206) form data aggregator 210 that collects and aggregates data from a plurality of disparate data sources. Specifically, data aggregator 210 generates a plurality of longitudinal data profiles, as described herein. Further, data analyzer 215 analyzes the longitudinal data profiles (e.g., to determine efficacy of a patient support program associated with a prescription product or to compare adherence levels between patients).

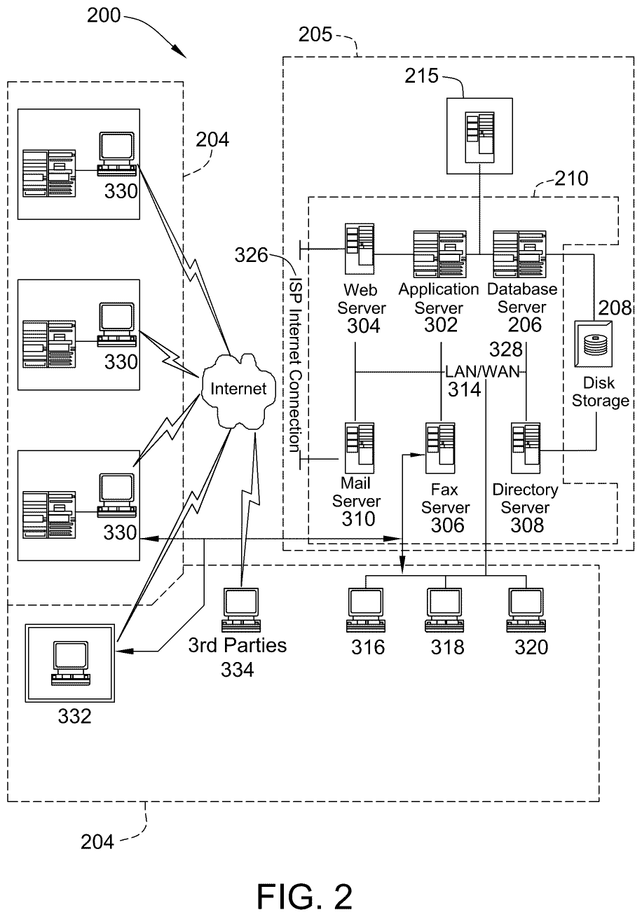

FIG. 2 is an expanded block diagram of an example embodiment of a server architecture of healthcare data processing system 200 in accordance with one embodiment of the present disclosure. Healthcare data processing system 200 includes client systems 204 and data hub 205 having data aggregator 210 and data analyzer 215. Server system 202 includes database server 206, an application server 302, a web server 304, a fax server 306, a directory server 308, and a mail server 310. Database 208 (e.g., a disk storage unit), is coupled to database server 206 and directory server 308. Servers 206, 302, 304, 306, 308, and 310 are coupled in a local area network (LAN) 314. In addition, a system administrator's workstation 316, a user workstation 318, and a supervisor's workstation 320 are coupled to LAN 314. Alternatively, workstations 316, 318, and 320 are coupled to LAN 314 using an Internet link or are connected through an Intranet.

Each workstation, 316, 318, and 320, is a personal computer having a web browser. Although the functions performed at the workstations typically are illustrated as being performed at respective workstations 316, 318, and 320, such functions can be performed at one of many personal computers coupled to LAN 314. Workstations 316, 318, and 320 are illustrated as being associated with separate functions only to facilitate an understanding of the different types of functions that can be performed by individuals having access to LAN 314.

Server system 202 is configured to be communicatively coupled to various entities (e.g., data sources, as described in detail herein), including third parties 334 using an Internet connection 326. Server system 202 is also communicatively coupled to healthcare data analyzer 215. In some embodiments, healthcare data analyzer 215 is integrated within server system 202. The communication in the example embodiment is illustrated as being performed using the Internet, however, any other wide area network (WAN) type communication can be utilized in other embodiments, e.g., the systems and processes are not limited to being practiced using the Internet. In addition, and rather than WAN 328, local area network 314 could be used in place of WAN 328.

In the example embodiment, any authorized individual or entity having a workstation 330 may access system 200. At least one of the client systems includes a manager workstation 332 located at a remote location. Workstations 330 and 332 include personal computers having a web browser. Also, workstations 330 and 332 are configured to communicate with server system 202. Furthermore, fax server 306 communicates with remotely located client systems, including a client system 332, using a telephone link. Fax server 306 is configured to communicate with other client systems 316, 318, and 320 as well.

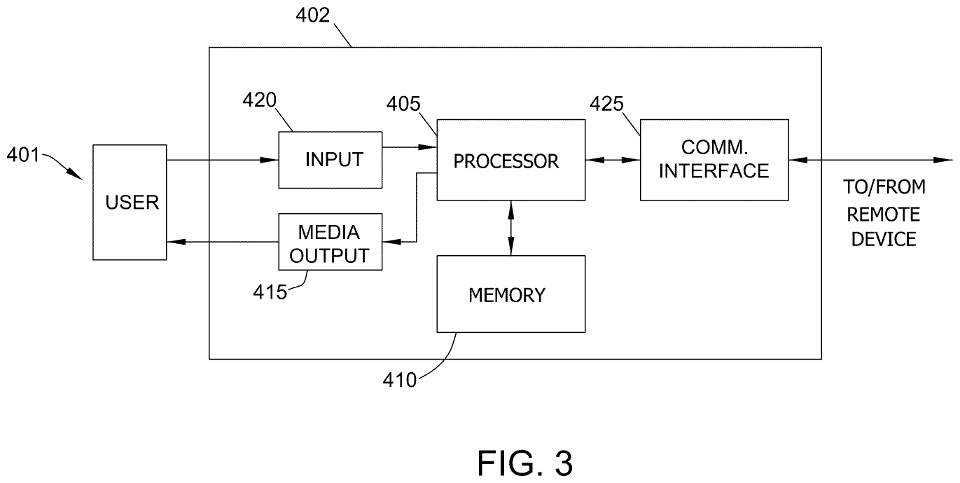

FIG. 3 illustrates an example configuration of a data source computing device 402 operated by a user 401. Data source computing device 402 may include, but is not limited to, client systems (“client computing devices”) 204, 316, 318, and 320, workstation 330, and manager workstation 332 (shown in FIG. 2).

Data source computing device 402 includes one or more processors 405 for executing instructions. In some embodiments, executable instructions are stored one or more memory devices 410. Processor 405 may include one or more processing units (e.g., in a multi-core configuration). One or more memory devices 410 are any one or more devices allowing information such as executable instructions and/or other data to be stored and retrieved. One or more memory devices 410 may include one or more computer-readable media.

Data source computing device 402 also includes at least one media output component 415 for presenting information to user 401. Media output component 415 is any component capable of conveying information to user 401. In some embodiments, media output component 415 includes an output adapter such as a video adapter and/or an audio adapter. An output adapter is operatively coupled to processor 405 and operatively coupleable to an output device such as a display device (e.g., a liquid crystal display (LCD), organic light emitting diode (OLED) display, cathode ray tube (CRT), or “electronic ink” display) or an audio output device (e.g., a speaker or headphones).

In some embodiments, data source computing device 402 includes an input device 420 for receiving input from user 401. Input device 420 may include, for example, a keyboard, a pointing device, a mouse, a stylus, a touch sensitive panel (e.g., a touch pad or a touch screen), a gyroscope, an accelerometer, a position detector, or an audio input device. A single component such as a touch screen may function as both an output device of media output component 415 and input device 420.

Data source computing device 402 may also include a communication interface 425, which is communicatively coupleable to a remote device such as server system 202. Communication interface 425 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in one or more memory devices 410 are, for example, computer-readable instructions for providing a user interface to user 401 via media output component 415 and, optionally, receiving and processing input from input device 420. A user interface may include, among other possibilities, a web browser and client application. Web browsers enable users, such as user 401, to display and interact with media and other information typically embedded on a web page or a website from server system 202. A client application allows user 401 to interact with a server application from server system 202 or a web server.

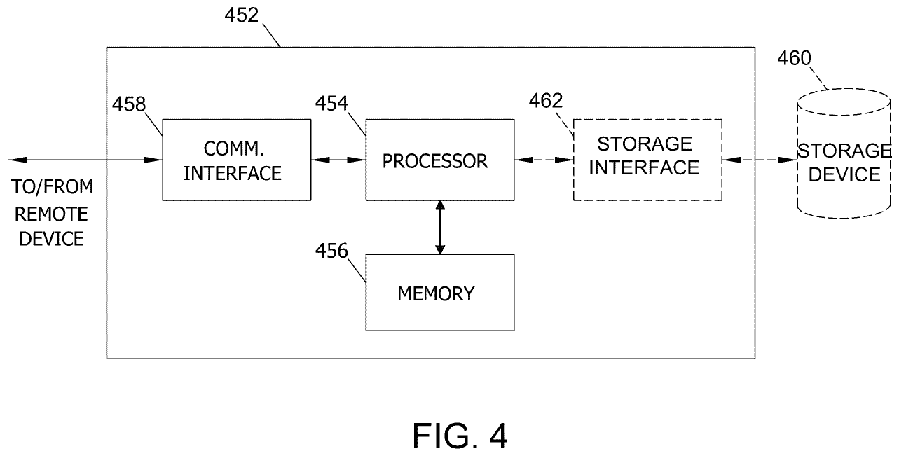

FIG. 4 illustrates an example configuration of a server computing device 452 such as server system 202 (shown in FIGS. 1 and 2). Server computing device 452 may include, but is not limited to, database server 206, application server 302, web server 304, fax server 306, directory server 308, and mail server 310. Server computing device 452 is also representative of healthcare data analyzer 215.

Server computing device 452 includes one or more processors 454 for executing instructions. Instructions may be stored in one or more memory devices 456, for example. One or more processors 454 may include one or more processing units (e.g., in a multi-core configuration).

One or more processors 454 are operatively coupled to a communication interface 458 such that server computing device 452 is capable of communicating with a remote device such as data source computing device 402 or another server computing device 452. For example, communication interface 458 may receive requests from client systems 204 via the Internet or another network, as illustrated in FIGS. 1 and 2.

One or more processors 454 may also be operatively coupled to one or more storage devices 460. One or more storage devices 460 are any computer-operated hardware suitable for storing and/or retrieving data. In some embodiments, one or more storage devices 460 are integrated in server computing device 452. For example, server computing device 452 may include one or more hard disk drives as one or more storage devices 460. In other embodiments, one or more storage devices 460 are external to server computing device 452 and may be accessed by a plurality of server computing devices 452. For example, one or more storage devices 460 may include multiple storage units such as hard disks or solid state disks in a redundant array of inexpensive disks (RAID) configuration. One or more storage devices 460 may include a storage area network (SAN) and/or a network attached storage (NAS) system. In some embodiments, one or more storage devices 460 may include database 208.

In some embodiments, one or more processors 454 are operatively coupled to one or more storage devices 460 via a storage interface 462. Storage interface 462 is any component capable of providing one or more processors 454 with access to one or more storage devices 460. Storage interface 462 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing one or more processors 454 with access to one or more storage devices 460.

One or more memory devices 410 and 456 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are example only, and are thus not limiting as to the types of memory usable for storage of a computer program.

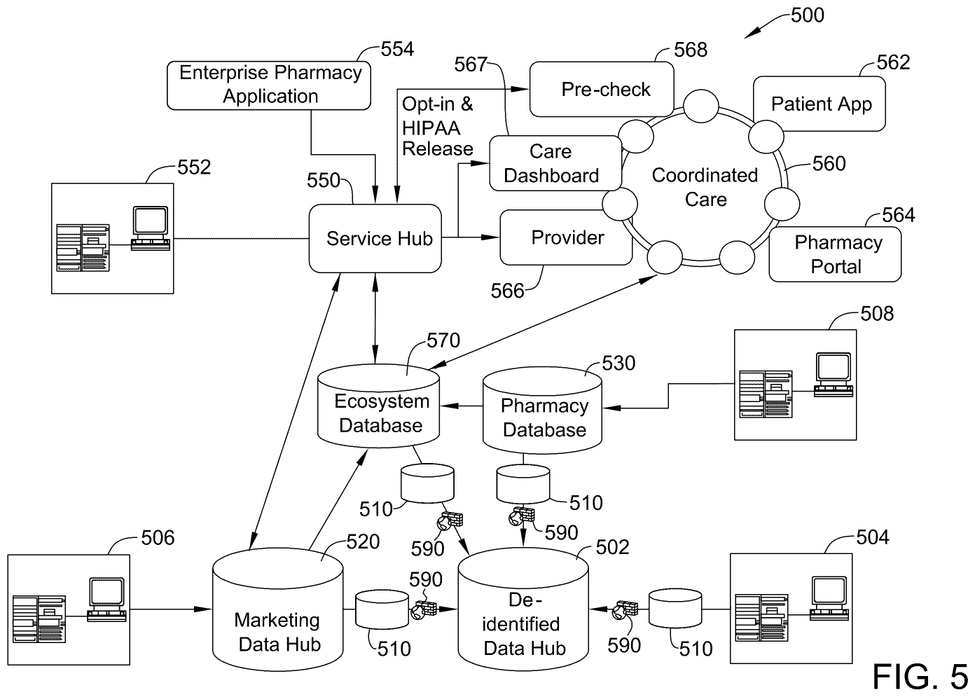

FIG. 5 illustrates an example architecture of a healthcare data processing system 500, such as healthcare data processing system 200 (shown in FIGS. 1 and 2). In the example embodiment, system 500 includes a central data hub 502, such as data hub 205 (shown in FIGS. 1 and 2). As described herein, central data hub 502 collects data from a plurality of disparate data sources, such as client systems 204 (shown in FIGS. 1 and 2). In the example embodiment, central data hub 502 generates longitudinal data profiles based on the collected data. Each longitudinal data profile includes healthcare data collected from multiple data sources for a particular patient. The data is (i) collected, (ii) encrypted to de-identify the particular patient, (iii) assigned an anonymous ID, and (iv) formatted into a single format so that it can be stored in central data hub 502, wherein the formatting includes organizing the data into a predetermined structure such as organizing the data in chronological order, and/or based on temporal information.

The data sources may include claims data sources 504, services marketing, and customer relationship management (CRM) data sources (collectively referred to herein as “services data sources” 506), and pharmacy data sources 508. Alternatively, the data sources may include other types of data sources. In some embodiments, a patient application 562 (e.g., a software application) receives input from a patient related to patient adherence, as described in detail below. Patient application 562 may function as a claims data source 504, services data source 506, and/or pharmacy data source 508. As described herein, data may flow differently from each type of data source (e.g., claims data sources 504, services data sources 506, and pharmacy data sources 508) to central data hub 502. However, in the example embodiment, a syndicated data provider 510 encrypts the data from each of the disparate data sources, as described herein. Accordingly, because the same encryption algorithm is used for data from disparate data sources, when encrypted data arrives at central data hub 502 from one data source, that encrypted data can be linked or matched with other encrypted data from other data sources. The data from each of the data sources may be provided automatically (e.g., periodically or instantaneously whenever the data is updated) and/or in response to a request from central data hub 502. Further, the party operating central data hub 502 may specify to syndicated data provider 510 what types of data it wants to receive at central data hub 502.

In the example embodiment, claims data sources 504 are associated with data partners that typically store, process, and/or have access to insurance claims data associated with medical or pharmaceutical insurance coverage. These data partners may provide particular types of data to central data hub 502. Claims data sources 504 may include, for example a benefits verifier. For example, during the prescription process, the benefits verifier may receive a benefits request from an HCP and, in response, provide a benefits summary that details what insurance benefits the patient is entitled to.

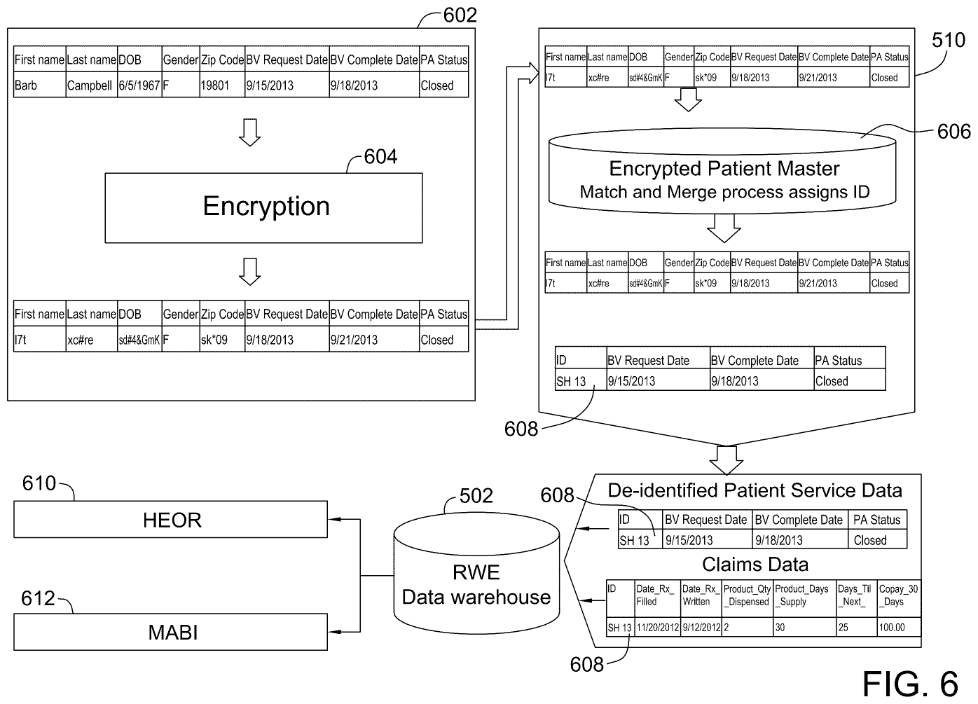

FIG. 6 is a flow diagram illustrating an example data flow from a benefits verifier 602 to central data hub 502. In the example embodiment, benefits verifier 602 is part of claims data source 504. In this example, the data provided by benefits verifier 602 includes a patient first name, patient last name, patient date of birth, patient gender, patient zip code, benefits verification request date (e.g., the date the benefits request was received at benefits verifier 602), a benefits verification complete date (e.g., the date benefits verifier 602 provided the benefits summary), and prior authorization status (e.g., whether any necessary prior authorization form for the prescription product has been retrieved by the benefits verifier). Additionally or alternatively, other suitable patient information may be used.

As shown in FIG. 6, the benefits verifier data is encrypted, at benefits verifier 602, using an encryption module 604. Alternatively, the benefits verifier data may be transmitted to an encryption module external to benefits verifier 602. In the example embodiment, the data is encrypted in accordance with the encryption algorithm of syndicated data provider 510. For example, encryption module 604 may be provided by syndicated data provider 510. The encrypted benefits verifier data is transmitted from benefits verifier 602 to syndicated data provider 510. Syndicated data provider 510 assigns an anonymous ID 608 to the encrypted benefits verifier data, using an encrypted patient master 606. In the example embodiment, encrypted patient master 606 is a roster that includes a list of patient identifiers and an anonymous ID 608 associated with each patient identifier. The patient identifier may be patient information (e.g., name, address, etc.) in an encrypted form or an un-encrypted form. Syndicated data provider 510 determines the patient identifier from the encrypted data, and assigns the appropriate anonymous ID 608 if the patient identifier is already listed on encrypted patient master 606. If the patient identifier is not already listed on encrypted patient master 606 (e.g., for new patients), syndicated data provider 510 generates a new, unique anonymous ID 608.

Accordingly, in the example embodiment, a unique anonymous ID 608 is assigned to a particular patient, and other data received by central data hub 502 for this same patient will be assigned the same anonymous ID 608 such that all data associated with this particular patient is able to be linked together, even though the patient name is no longer identified. Once anonymous ID 608 is assigned to the encrypted data, the encrypted data may be referred to as “de-identified data.” In some embodiments, syndicated data provider 510 may also perform additional masking of certain data elements (e.g., prescription number) to generate the de-identified data.

Syndicated data provider 510 also receives claims data from a claims switch (not shown in FIG. 6). As used herein, a claims switch is a computing device associated with an entity that routes claims from a pharmacy to a payer (e.g., an insurance provider). The claims data may include, for example, date the prescription was filled, date the prescription was written, quantity of the drug dispensed, supply amount of the drug (e.g., 30 day supply), days until next prescription required, and co-pay amount for the prescription. The claims data is also assigned the anonymous ID by syndicated data provider 510. Accordingly, using the anonymous ID, syndicated data provider 510 matches the de-identified benefits verifier data with the claims data, and transmits the matched data to central data hub 502 (e.g., for analysis using an health economics outcomes research (HEOR) analytic framework 610 and/or a marketing analytics business intelligence (MABI) analytic framework 612). In the example embodiment, the claims data includes claims data for most, if not all, patients, including both patients that are participating in a patient support program and patients that are not participating in the patient support program.

The data flow for other claims data sources 504 is generally similar to the data flow for benefits verifier 602. Other claims data sources 504 may include, for example, a co-pay data source that provides co-pay data (e.g., co-pay amount, amount reimbursed to pharmacy, patient out of pocket responsibility, total amount of the processed claim, etc.), a prior authorization data source that provides prior authorization data (e.g., data from a prior authorization form library, data related to electronic prior authorization forms, etc.), an employer data source that provides work productivity data (e.g., days missed, hours worked, etc.), and a clinical data source that provides electronic health record (EHR) data, medication fill history data, and/or medical history data. EHR data may include data related to allergies, appointments, medications, physicians, vaccinations, order requests, patient demographics, patient problems including diagnosis, patient compliance, patient education/training on medications, etc. Medical history data may be entered during a patient-HCP encounter, and may include data related to condition history (e.g., primary and secondary diagnoses), medication examination (e.g., HCP name, date HCP visited, vaccinations, screenings, etc.), lab results, medical procedures, national drug code (NDC) for drug prescribed, dosing, etc.

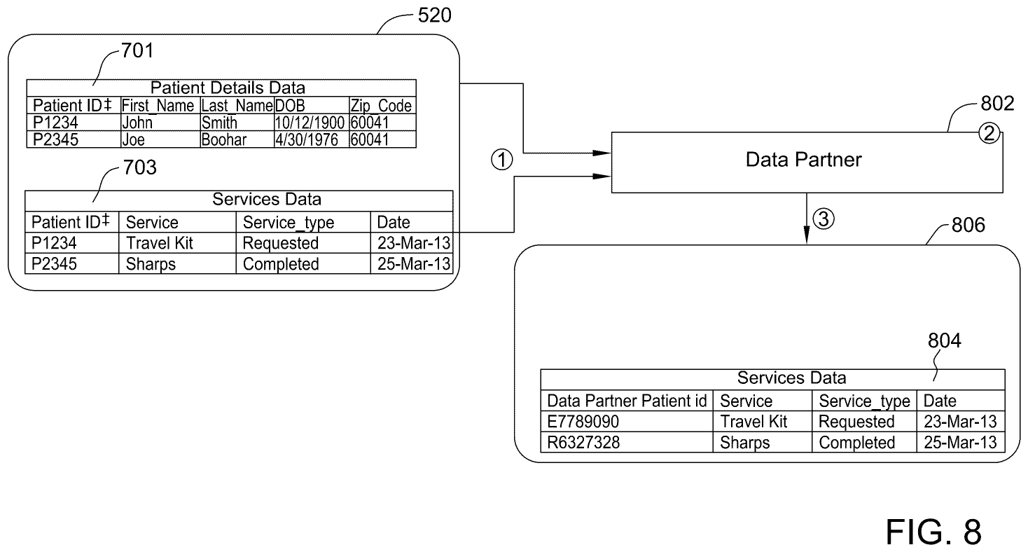

As described herein, in the example embodiment, for claims data sources 504, services data sources 506, and pharmacy data sources 508, data is processed by transmitting data to central data hub 502 via syndicated data provider 510. In contrast, for some data partners, data is integrated at a marketing database and sent to a data analysis party separately as an aggregated dataset, as described below in relation to FIG. 8.

In the example embodiment, as shown in FIG. 5, central data hub 502 is separated from syndicated data provider 510 by a firewall 590 to ensure that a party operating central data hub 502 is restricted from accessing unencrypted or identified data. Further, data communication between syndicated data provider 510 and central data hub 502 is one-way, such that central data hub 502 receives data from syndicated data provider 510 but may be restricted from transmitting data to syndicated data provider 510. This ensures that the operator of central data hub only has access to de-identified data. In some embodiments, a filter or control may be configured to allow adjustable limited data communication between syndicated data provider 510 and central data hub 502.

Referring back to FIG. 5, services data sources 506 are data sources associated with data partners that typically store, process, and/or have access to data related to one or more services provided in association with the prescription product. Services data sources 506 provide data to a marketing database 520. The data is generally related to services provided to a patient as part of the prescription process. Marketing database 520 ultimately provides the data to central data hub 502, as described herein. Services data sources 506 may include, for example, an adherence tracking data source that provides adherence tracking data (e.g., data indicating that the patient has taken a dose of a prescribed medication), a customer relationship management (CRM) data source that provides CRM data (e.g., data from a CRM program that assists the patient during the prescription process), a healthcare coordinator data source that provides data related to a healthcare coordinator (e.g., live or virtual) that guides the patient through the prescription process, a call center data source that provides call center data, and a nursing data source that provides nursing data (e.g., data related to injection training given to the patient). CRM data may include, for example, a list of services (e.g., medication reminder, co-pay savings card, nurse injection training, etc.) that a patient has subscribed to, the patient's utilization of those services, marketing campaigns, etc. For instance, CRM data may include a program name, a campaign name, a patient kit name, a marketing objective, a service type, and/or a service opt-in date.

Data from the one or more services data sources 506 is received and stored in marketing database 520. Marketing database 520 may be operated, for example, by a customer service and/or marketing entity. Notably, the data stored in marketing database 520 is generally identified (e.g., non-encrypted, non-anonymous data). Accordingly, the data stored in marketing database 520 may be de-identified before it is received and stored in central data hub 502.

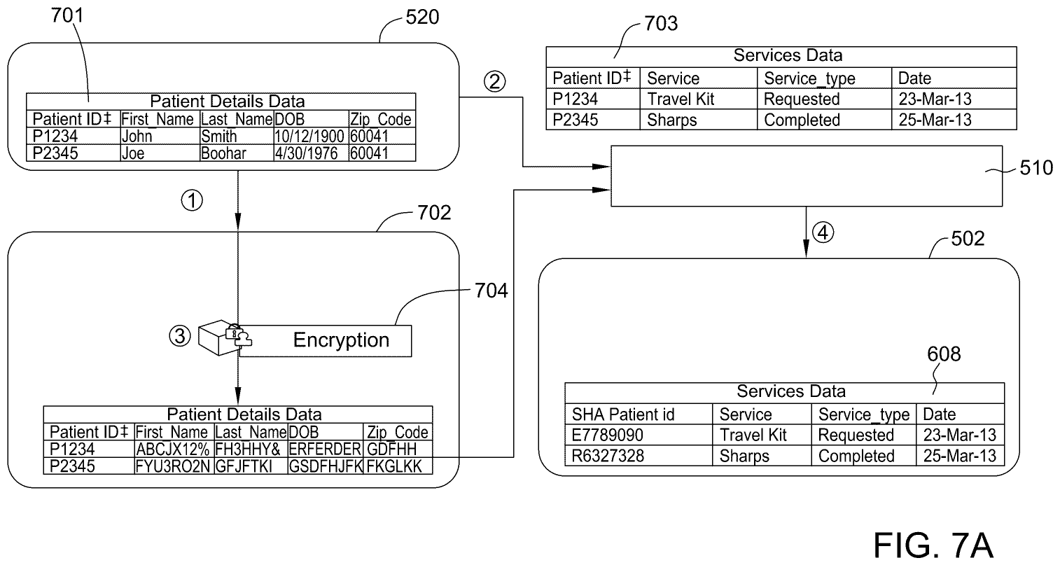

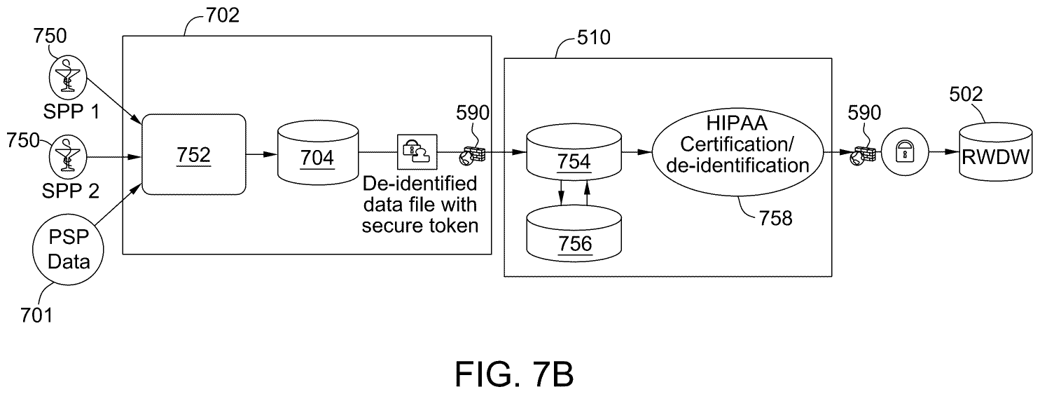

FIG. 7A is an example flow diagram illustrating the data flow from marketing database 520 to central data hub 502. In the example embodiment, the data stored in marketing database 520 includes patient details data 701 and services data 703. Patient details data 701 may include, for example, CRM data associated with a marketing campaign, such as a patient ID (which may be a separate identifier from anonymous ID 608 assigned by syndicated data provider 510), patient first name, patient last name, patient date of birth, and patient zip code. Services data 703 may include, for example, data related to a patient support program and patient utilization of the program, such as the patient ID, service provided (e.g., travel kit, injection materials, etc.), service type (e.g., service requested, service completed), and date.

As shown in FIG. 7A, because marketing database 520 may not include an encryption module, in the example embodiment, patient details data 701 is sent from marketing database 520 to a claims switch 702. Claims switch 702 uses an encryption module 704 to encrypt patient details data 701 in accordance with the encryption algorithm of syndicated data provider 510. The encrypted patient details data is transmitted from claims switch 702 to syndicated data provider 510.

Services data 703 is sent from marketing database 520 to syndicated data provider 510 via a secure file transfer protocol (FTP) process. In the example embodiment, services data 703 is not identifiable data, and accordingly, does not need to be encrypted by claims switch 702. Syndicated data provider 510 assigns anonymous ID 608 to the encrypted patient data, using encrypted patient master 606 (shown in FIG. 6). Further, for the services data, syndicated data provider 510 replaces the patient ID with anonymous ID 608. The encrypted patient data with the anonymous ID and the services data with the anonymous ID are merged and sent to central data hub 502.

FIG. 7B is another example flow diagram illustrating the data flow from marketing database 520 to central data hub 502. As shown in FIG. 7B, claims switch 702 receives patient details data 701. Claims switch 702 may be an actual claims switch, a data provider, or a data consolidator in the flow of FIG. 7B. Claims switch 702 may also receive data from one or more specialty pharmacies 750. Claims switch 702 may, for example, secure exchanges of clinical, financial, and administrative information between patients, providers, payers, and pharmacies. The data is received at a data reception component 752 of the claims switch/data provider/data consolidator and provided to encryption module 704. Encryption module 704 de-identifies the data. In the example embodiment, encryption module 704 uses specific patient identifiable data within each data record to generate a series of unique de-identified patient tokens. These tokens (and the de-identified data) are then transmitted through a firewall 590 to a database 754 of syndicated data provider 510 using a secure FTP process.

Syndicated data provider 510 performs patient matching by comparing the de-identified patient tokens against a patient master 756, such as patient master 606 (shown in FIG. 6), to assign the appropriate anonymous ID to each record. The tokens may then be removed from the dataset and stored in a separate secure environment. With the anonymous ID assigned, in the example embodiment, a HIPAA statistician performs a review 758 to certify that the resulting dataset meets HIPAA standards for patient de-identification. After review 758 is complete, the de-identified data and associated anonymous ID are transmitted through a firewall 590 to central data hub 502. As described above in reference to FIG. 5, firewall 590 facilitates ensuring that a party operating central data hub 502 is unable to access unencrypted or identified data.

FIG. 8 is an example flow diagram illustrating data flow for data partners 802 that do not provide data to central data hub 502. Specifically, as noted above, some data partners 802 (e.g., employer data sources) integrate their data with data from marketing database 520 to create an aggregated data set 804. Aggregated data set 804 may not be transmitted to central data hub 502, and may instead be separately delivered to a computer system of a data analysis party 806. Data analysis party 806 may be, for example, an operator of central data hub 502.

In the embodiment shown in FIG. 8, patient details data 701 and services data 703 is sent from marketing database 520 to a data partner 802. Data partner 802 matches patient details data 701 and services data 703 with an internal patient master that is different from encrypted patient master 606. Data partner 802 may encrypt patient details data 701 and services data 703 using the encryption algorithm provided by syndicated data provider 710 or using a separate encryption algorithm.

Data partner 802 also replaces the patient ID in patient details data 701 and services data 703 with a data partner patient ID using the internal patient master. The patient details data 701 and services data 703 (with the substituted data partner patient ID) is integrated with data from data partner 802 to form aggregated data set 804, and aggregated data set 804 is transmitted to data analysis party 806 via secure FTP. Aggregated data set 804 may include data aggregated by, for example, a patient zip code, a predetermined period of time (e.g., monthly), etc.

Referring back to FIG. 5, pharmacy data sources 508 are data sources that provide data to an aggregated pharmacy database 530. In the example embodiment, pharmacy data sources 508 may transmit pharmacy data to pharmacy database 530 under an agreement. Pharmacy data may include, for example, shipment tracking data (e.g., tracking shipments of the prescription product) data related to a grace filling of the prescription product, fill history data, prescription transfer data, benefits verification data, prior authorization data, and insurance data. For example, when a patient is prescribed a prescription product, the patient's insurance company may mandate that a predetermined pharmacy (e.g., a mail-order pharmacy) fill the prescription. However, before the mandated pharmacy begins filling the prescription, another pharmacy (e.g., an independent pharmacy) may provide an initial “grace filling” for the prescription to ensure the patient gets an initial quantity of the prescription product relatively quickly. Data from the pharmacy data sources may also include, for example, data related to record information (e.g., date and time a record was generated at the pharmacy data source, a pharmacy national provider identifier (NPI) number, etc.), patient demographics (e.g., patient referral source, date and time patient referral was received at the pharmacy data source, etc.), patient services (e.g., whether a patient was offered access to a support program, whether a patient is currently a member of the support program, etc.), status information (e.g., date when a prior authorization expires, date and time a patient was assigned a prior authorization status, etc.), HIPAA authorization (e.g., whether patient executed HIPAA authorization, date HIPAA authorization was discussed with patient, etc.), prescriber demographics (e.g., prescriber name, prescriber state license number, etc.), prescription/shipment information (e.g., date prescription was written, number of refills remaining, etc.), insurance information (e.g., name of pharmacy benefits manager, type of benefit coverage, etc.), claim information (e.g., type of coverage applied to claim, policy expiration date, etc.), transfer information (e.g., name of pharmacy receiving transfer, date of beginning of patient transfer, etc.), and other information (e.g., pertinent notes on patient or service delivery).

The pharmacy data includes data covered by the Health Insurance Portability and Accountability Act of 1996 (referred to herein as “HIPAA data”) and/or any other healthcare and/or privacy regulations. In the example embodiment, at pharmacy database 530, the HIPAA data is encrypted, using an encryption module, in accordance with the encryption algorithm of syndicated data provider 510. Alternatively, pharmacy data sources 508 may include encryption modules that encrypt the pharmacy data before transmission to pharmacy database 530. The encrypted HIPAA data is sent to syndicated data provider 510 and matched with claims data from the claims switch, and the matched data is sent to central data hub 502 for storage and analysis.

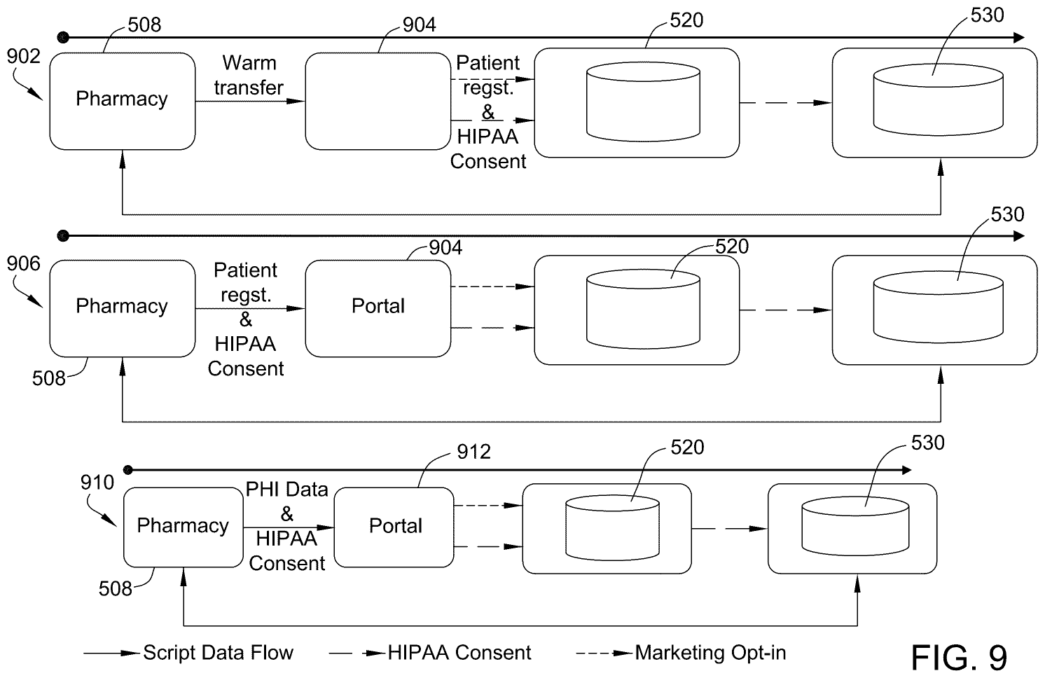

FIG. 9 is an example flow diagram showing multiple embodiments of an example data flow from a pharmacy data source 508 to marketing database 520 and pharmacy database 530. In a first data flow 902, pharmacy data source 508 warm transfers a patient prescribed a prescription to a call center 904 (e.g., by transferring the patient from a telephone call with a pharmacy to a telephone call with the call center). An operator at call center 904 registers the patient in a marketing program (by obtaining a marketing opt-in) and obtains regulatory (e.g., HIPAA) consent from the patient. Data related to the marketing opt-in and regulatory (e.g., HIPAA) consent is then sent to marketing database 520, and marketing database 520 periodically (e.g., daily) provides pharmacy database 530 with a list of patients who have provided regulatory (e.g., HIPAA) consent. Pharmacy database 530 retrieves data from pharmacy data source 508 for patients who have provided regulatory (e.g., HIPAA) consent, and releases that data in an identifiable form to an ecosystem database (not shown in FIG. 9).

In a second data flow 906, pharmacy data source 508 obtains a patient's regulatory (e.g., HIPAA) consent and marketing opt-in via a pharmacy portal 908 managed by the operator of central data hub 502, and data related to obtained regulatory (e.g., HIPAA) consents and marketing opt-ins is sent to marketing database 520 periodically (e.g., daily). Marketing database 520 then periodically (e.g., daily) provides pharmacy database 530 with a list of patients who have provided regulatory (e.g., HIPAA) consent, and pharmacy database 530 retrieves prescription data from pharmacy data source 508 based upon the regulatory (e.g., HIPAA) consents.

In a third data flow 910, pharmacy data source 508 obtains a patient's regulatory (e.g., HIPAA) consent and marketing opt-in via a pharmacy portal 912 managed by pharmacy data source 508, and data related to obtained regulatory (e.g., HIPAA) consents and marketing opt-ins is sent to marketing database 520 periodically (e.g., daily). Marketing database 520 then periodically (e.g., daily) provides pharmacy database 530 with a list of patients who have provided regulatory (e.g., HIPAA) consent, and pharmacy database 530 retrieves prescription data from pharmacy data source 508 based upon the regulatory (e.g., HIPAA) consents. First, second, and third data flows, 902, 906, and 908 all enable pharmacy database 530 to retrieve data from pharmacy data sources 508 based upon acquired regulatory (e.g., HIPAA) consents.

Referring back to FIG. 5, in this embodiment, system 500 includes a service hub 550 implemented, for example, using a messaging layer. Service hub 550 may be, for example, a server system, such as server system 202 (shown in FIGS. 1 and 2). Service hub 550 receives data from at least one service provider 552. Service providers 552 may be, for example, client systems, such as client systems 204 (shown in FIGS. 1 and 2). Service providers 552 may include, for example, an electronic health record provider that supplies electronic prior authorization data and fill history data, a reimbursement provider that provides electronic benefits verification data, a data consolidator that provides electronic medical/pharmacy benefits verification data, benefits eligibility and medication fill history data, and medical claims/services history data, a co-pay provider that provides co-pay data, and a co-pay eligibility provider that provides co-pay eligibility information.

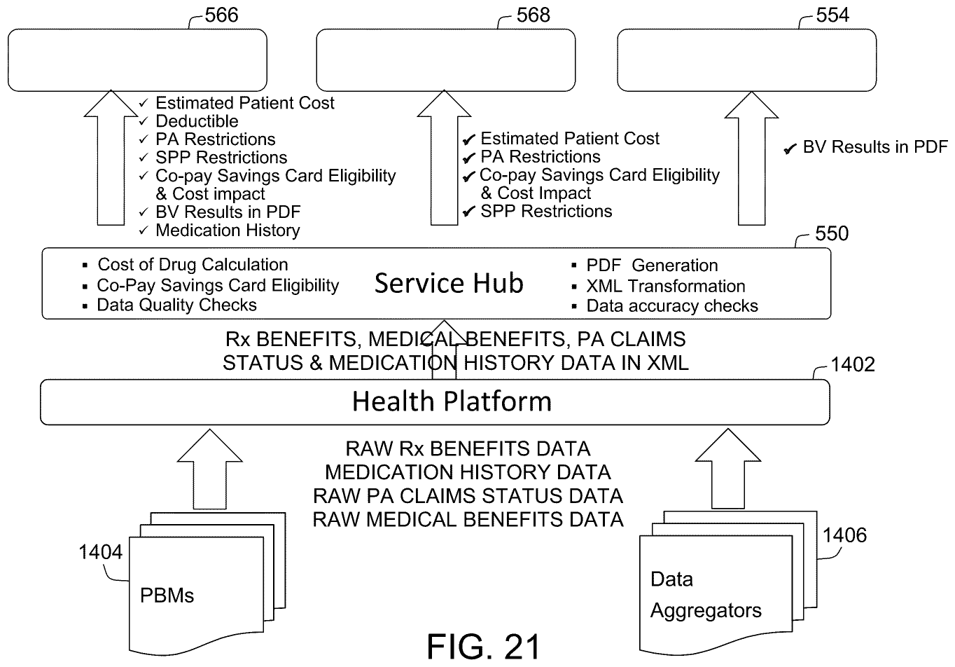

The data consolidator may be a comprehensive report platform that provides a range of services, including a secure, reliable, scalable, and regulatory (e.g., HIPAA) compliant reports system. The data consolidator connects to payers and data aggregators (e.g., the data sources and databases described herein) to deliver pharmacy eligibility information, pharmacy benefits information, medical benefits information, and medication history information for patients. In the example embodiment, the data consolidator translates raw data from a source into structured data (e.g., XML data), and delivers the structured data to various applications via service hub 550, as described below in relation to FIG. 21. Further, service hub 550 may apply business rules to calculate cost of drug for the patient, calculate co-pay savings card eligibility, generate PDF documents, etc., as described herein. In the example embodiment, service hub 550 then delivers processed data in a defined structured data schema to various applications.

Service hub 550 may also receive data from an enterprise pharmacy application 554. Data received from enterprise pharmacy application 554 may include, for example, data related to a benefits verification request, prior authorization forms, and/or benefits verification results.

As shown in FIG. 5, marketing service data is exchanged between service hub 550 and marketing database 520. The marketing service data may include, for example, patient demographic data (e.g., patient name, gender, address, etc.), patient consent data (e.g., date HIPAA consent given, date HIPAA consent expires, etc.), patient services data (e.g., service name, service opt-in date, etc.), diagnosis data (e.g., diagnosis name), co-pay savings card data (e.g., cardholder ID, prescription group of co-pay savings card, etc.), healthcare coordinator data (e.g., name of healthcare coordinator, date when healthcare coordinator began working with the patient, date when healthcare coordinator visited patient, etc.), on call nurse data (e.g., call date, call duration, etc.), patient survey data (e.g., date when survey taken, question text, etc.), service utilization data (e.g., service name, date service requested, etc.), patient insurance date (e.g., payer identification, insurance name, etc.), adverse event data (e.g., event description, event reported date, etc.), and benefits summary data (e.g., drug coverage status, co-pay amount, etc.). Further, service hub 550 exchanges data with components of a coordinated care platform 560. Coordinated care platform 560 streamlines the prescription process and enables users to track the status of a particular prescription.

In this embodiment, coordinated care platform 560 includes patient application 562 (e.g., a software application) that enables the patient to manage and track their condition and treatment regimen, a pharmacy portal 564 that enables a pharmacy to manage and track prescriptions, a healthcare provider (HCP) portal 566 that enables an HCP to manage and track prescriptions, a care dashboard 567 that enables users to quickly and easily track the status of a prescription for a patient, and a pre-check application 568 (e.g., a software application) that enables a prospective patient to quickly determine his or her eligibility for a prescription product. In some embodiments, pre-check application 568 is accessible to the patient through patient application 562. For example, a prospective patient may input their first and last name, date of birth, gender, and zip code into a web-based interface, and receive an estimated cost and/or co-pay eligibility determination for a particular prescription product in return. Pre-check application 568 may also require a prospective patient to complete a HIPAA opt-in and a marketing opt-in before returning prior authorization status (e.g., an indication of whether the prescription requires a prior authorization form), a specialty pharmacy (SPP) status (e.g., an indication of what SPP would be used to fill the prescription), the estimated cost, and/or co-pay eligibility information. An ecosystem database 570 stores data related to operation of coordinated care platform 560. In this embodiment, ecosystem database 570 receives data from pharmacy database 530 (e.g., a HIPAA release) and marketing database 520 (e.g., patient details data 701) for use by coordinated care platform 560. Further, ecosystem database 570 may transmit data received from service hub 550 and/or coordinated care platform 560 to central data hub 502 via syndicated data provider 510.

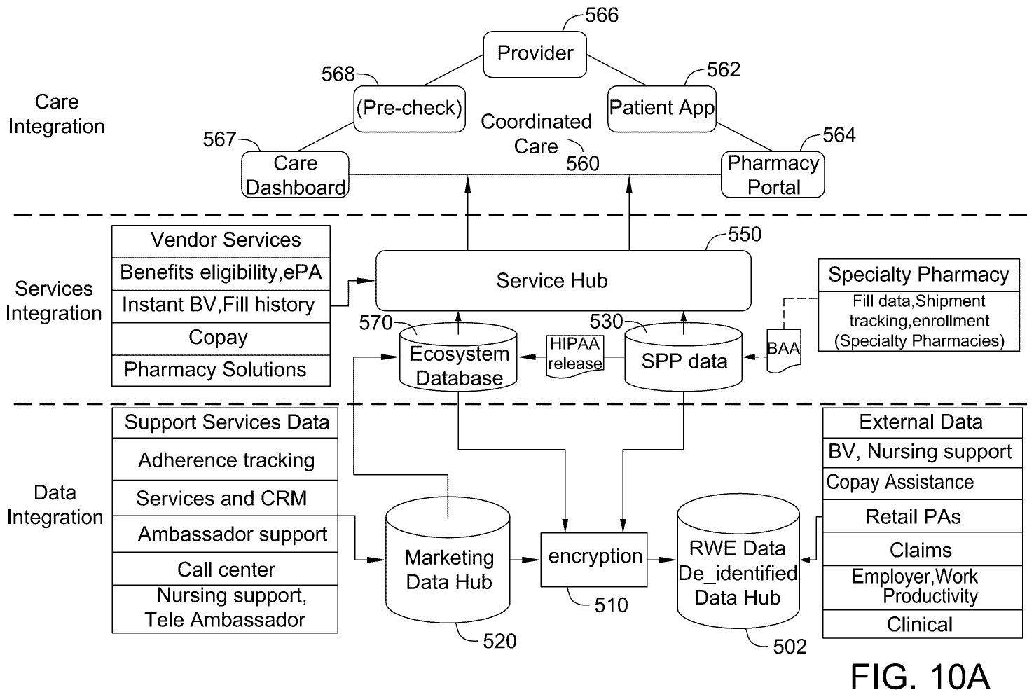

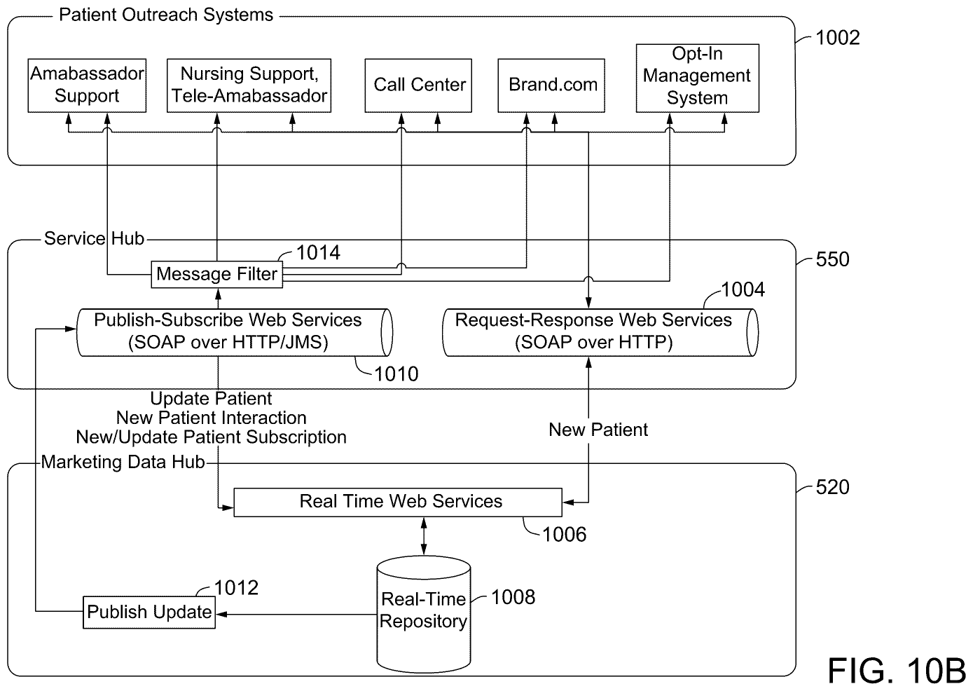

FIG. 10A illustrates data flow within system 500 (shown in FIG. 5) in the example embodiment. As shown in FIGS. 5 and 10, ecosystem database 570 provides data to central data hub 502 via syndicated data provider in the example embodiment. Ecosystem database 570 may provide, for example, data acquired at pharmacy portal 564 (e.g., HIPAA consents, marketing opt-ins, etc.) and data acquired through patient application 562 (e.g., survey results data, medication consumption data, subscription in a medication reminder program, marketing opt-ins, self-reported patient adherence, etc.).

FIG. 10B illustrates real-time data integration within system 500 (shown in FIG. 5) in the example embodiment. Specifically, real-time data integration is provided between one or more patient outreach systems 1002 (e.g., patient support programs) and marketing data hub 520. Patient outreach systems 1002 may include, for example, an ambassador support program, a nursing support/tele-ambassador program, a call center, a website associated with a prescription product, and an opt-in management system (e.g., for managing patient opt-ins into patient support programs). Alternatively, patient outreach systems 1002 may include any suitable patient support programs.

As shown in FIG. 10B, to accomplish real-time data integration, patient outreach systems 1002 transmit new patient data (e.g., support program enrollment data) to marketing data hub 520 through service hub 550. Specifically, new patient data is transmitted to marketing data hub 520 using a request-response messaging channel 1004 of service hub 550. Request-response messaging channel 1004 may be implemented, for example, using simple object access protocol (SOAP) over HTTP. In the example embodiment, the new patient data is transmitted in real-time (e.g., the new patient data is forwarded to marketing data hub 520 at substantially the same time that the new patient data is input at one or more patient outreach systems 1002).

At marketing data hub 520, a real-time web services module 1006 receives the new patient data and stores it in a real-time repository 1008. The marketing data hub 520 also returns, via request-response messaging channel 1004, a patient ID (e.g., an alphanumeric identifier) to the patient outreach systems 1002. Any future data regarding the patient is communicated to marketing data hub 520 using the patient ID.

For example updated patient data, new interaction data for an existing patient, and new/updated subscription data for an existing patient may be transmitted to real-time web services module 1006 using a publish-subscribe messaging channel 1010 of service hub 550. Publish-subscribe messaging channel 1010 may be implemented, for example, using simple object access protocol (SOAP) over HTTP. The updated data received at real-time web services module 1006 is stored in real-time repository 1008.

In the example embodiment, based on the data stored in real-time repository 1008, a publish update module 1012 of marketing data hub 520 generates and transmits patient data updates to patient outreach systems 1002. Specifically, the patient data updates are transmitted via publish-subscribe messaging channel 1010 to a message filter 1014 that forwards the patient data updates to the appropriate patient outreach systems 1002.

The real-time data integration shown in FIG. 10A facilitates creating a complete patient record in marketing data hub 520 in real-time, and ensures patient data is represented consistently and accurately between marketing data hub 520 and patient outreach systems 1002. Accordingly, data for a new patient enrolled using one patient outreach system 1002 is made available to all other patient outreach systems 1002 substantially instantaneously. As compared to systems that periodically integrate data in batches, the real-time data integration described herein improves patient experience and reduces compliance risks by providing real-time and up-to-date patient data.

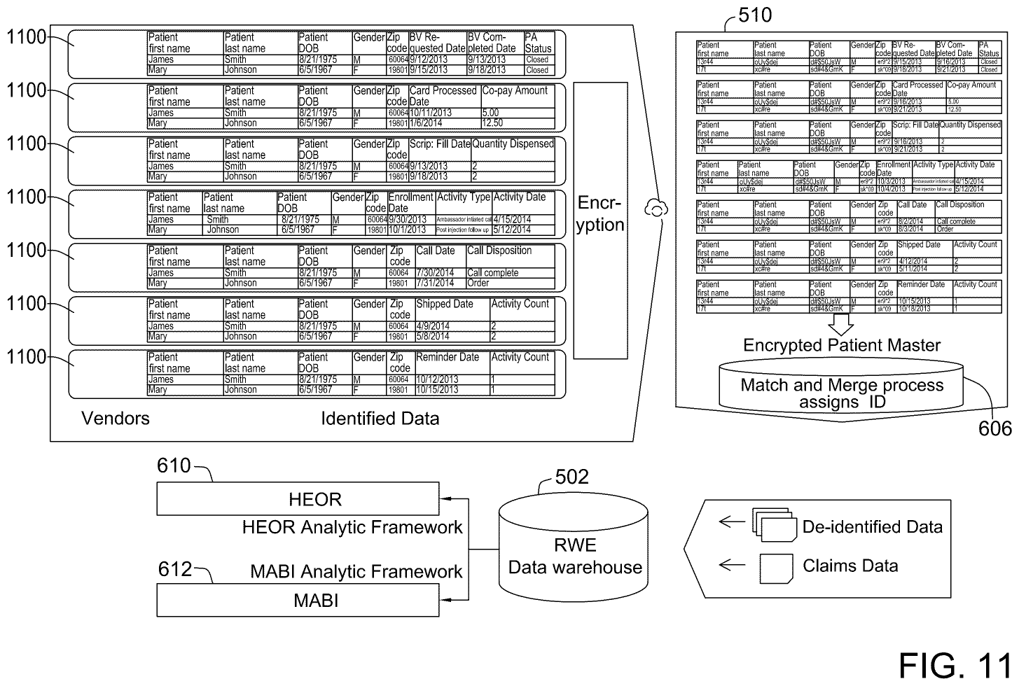

FIG. 11 illustrates a simplified overview of data flow in system 500 (shown in FIG. 5). As shown in FIG. 11, a plurality of vendors 1100 store, process, and/or have access to identified data. Each vendor 1100 may be, for example, one of claims data sources 504, services data sources 506, and pharmacy data sources 508 (all shown in FIG. 5). Accordingly, the identified data may be related to the prescription process (e.g., pharmacy data, co-pay data, benefits verification data, ambassador data, call center data, injection materials data, reminder data, etc.). The identified data is encrypted (e.g., at the vendor 1100) in accordance with the encryption algorithm of syndicated data provider 510, and then the encrypted data is passed to syndicated data provider 510. To fully de-identify the encrypted data, syndicated data provider 510 assigns anonymous ID 608 to the encrypted data, using encrypted patient master 606. The de-identified data, along with claims data, is then sent to central data hub 502.

Accordingly, central data hub 502 consolidates data from various vendors 1100 in a de-identified format. However, using anonymous ID 608, data from different vendors 1100 can be linked. By organizing and/or linking de-identified data from multiple vendors 1100, central data hub 502 creates a comprehensive, longitudinal data profile that includes all information related to the patient and the prescription that can be obtained from the various vendors 1100. This longitudinal data profile may include more information than may be independently provided by a single vendor 1100. For example, for a longitudinal data profile for a patient, central data hub 502 may allow filtering, sorting, and/or other manipulation of the collected data to generate one or more extracts for analysis, as described below.

In the example embodiment, central data hub 502 uses a standard layout for all data received at central data hub 502. This standard layout is provided to the various data sources, syndicated data provider 510, etc. to allow central data hub 502 to receive similar data feeds from multiple data providers in the same format. In the example embodiment, the standard layout includes key details associated with the data, such as date-related fields (e.g., effective dates, transaction dates, etc.). Further, when data is loaded into central data hub 502, a load date is added to every data record. These date-related fields facilitate organizing the longitudinal data profile (e.g., chronologically) when generating an extract, as described below.

In the example embodiment, after receiving a data feed in the standard layout, the data is loaded into a flexible layout, and the data is decomposed into ‘facts’ (e.g., what the data is) and ‘dimensions’ (e.g., who the data is associated with) and loaded into respective tables. This model allows the data to be loaded into a database of central data hub 502 relatively quickly, without requiring changes to be made to the loading process if there are changes/enhancements made to the standard layout.

A data quality management (DQM) system may be used to perform data quality checks on the data at both a file level and a table level. Specifically, in the example embodiment, after a data feed is received at central data hub 502, the data will go undergo file level checks to ensure the counts, file names, etc. are as expected. Subsequently, the data feed is loaded into a staging layer, where further quality checks are performed on the data. From the staging layer, an extract, transform, load (ETL) process loads the data into a flexible layer before decomposing the data into dimensions and facts. As noted above, using the flexible layer and decomposing the data allows the data to be loaded relatively quickly, without requiring changes to be made to the loading process if there are changes/enhancements made to the standard layout.

In one example, a longitudinal data profile stored in central data hub 502 includes a claim ID, anonymous ID, product, physician NPI number, physician key, pharmacy ID, primary insurance plan ID, date benefits verification requested, date benefit verification completed, benefit verification status, date nurse injection training ordered, date nurse injection training completed, nurse injection training location, healthcare coordinator enrollment start date, healthcare coordinator enrollment end date, injection container order date, activity count, injection container delivery date, co-pay card activation status, co-pay card use status, primary patient amount owed, primary insurance plan amount owed, co-pay amount, customary charge, fill type, dispense as written code, quantity dispensed, product supply in days, date prescription filled, and date prescription written. Although the data is de-identified, at least some patient demographic data is still included in the longitudinal data profile. For example, the longitudinal data profile may include the gender, birth year, household income, ethnicity, and/or geographic location (e.g., zip code) of the patient. Accordingly, longitudinal data profiles can be segmented (e.g., filtered) based on the demographic data when generating an extract. Alternatively, the longitudinal patient data profile may include any organization and/or type of information collected from the disparate data sources.

By analyzing a plurality of longitudinal data profiles, useful information about the overall prescription process may be obtained. That is, statistics may be generated from a plurality of longitudinal data profiles to determine the efficacy of one or more services, programs, or features implemented within the framework of system 500. For example, longitudinal data profiles for patients that participate in a patient support program may be compared with longitudinal data profiles for patients that do not participate in a patient support program to determine whether patients in the patient support program have an improved condition (e.g., resulting from improved adherence) for a particular prescription product. As used herein, a “patient condition” may include, for example, a patient outcome and/or healthcare costs. An improved condition may be based on, for example, a number of insurance claims made during a period of time that the patient participates in the patient support program, medical costs associated with the patient during a period of time that the patient participates in the patient support program, patient feedback, etc. Such information may be useful in demonstrating that the patient support program facilitates reducing overall medical expenses for patients. Longitudinal data profiles may be analyzed for both health economics outcomes research (HEOR) and marketing analytics business intelligence (MABI) purposes. The longitudinal data profiles may be analyzed, for example, using healthcare data analyzer 215 (shown in FIGS. 1 and 2).

In the example embodiment, healthcare data analyzer 215 is included within central data hub 502. Alternatively, healthcare data analyzer 215 may be separate from central data hub 502 and in communication with central data hub 502. In the example embodiment, healthcare data analyzer 215 receives one or more inputs from a user, and compares a plurality of longitudinal data profiles stored in central data hub 502 based on the inputs. Healthcare data analyzer 215 outputs (e.g., displays) results of the comparison for the user to review. For example, a user may provide inputs instructing healthcare data analyzer 215 to compare adherence data from longitudinal data profiles for patients using the prescription product who participate in a patient support program against longitudinal data profiles for patients using the prescription product who do not participate in the patient support program, or between multiple patients participating in the patient support program. Based on the results output by healthcare data analyzer 215, the user can determine whether participation in the patient support program is associated with an improvement in patient condition (e.g., improved patient satisfaction, fewer hospitalizations, reduced overall healthcare costs, fewer work days missed, etc.). For example, to illustrate an economic benefit of the patient support program, the analysis may output a cost differential between a patient who participates in the patient support program and a patient who does not participate in the patent support program.

Based on the desired analysis, a subset of longitudinal data profiles from central data hub 502 are provided to healthcare data analyzer 215 as an ‘extract’ from central data hub 502. For example, if one analysis is directed to the efficacy of a particular patient support program, all longitudinal data profiles for patients that are enrolled in the particular patient support program may be the extract provided to healthcare data analyzer 215. The extracts may be provided directly to healthcare data analyzer 215, or may be stored in a database (e.g., an FTP file server) that healthcare data analyzer 215 is able to access. Further, the extracts may be provided in any suitable format (e.g., as a spreadsheet file, manuscript file, etc.).

The extracts are generated based on user input received, for example at central data hub 502. The user input may indicate, for example, which longitudinal data profiles should be included in the extract (e.g., all longitudinal data profiles associated with a particular pharmacy benefit managers (PBM) or insurance provider). In another example, to generate the extract, longitudinal data profiles stored at central data hub 502 are segmented by patient characteristics (e.g., income level, age, geographic location, etc.) based on the user input. In the example embodiment, the user input may also indicate an organizational scheme for the longitudinal data profiles included in the extract. For example, the user input may specify that the data in each longitudinal data profile that is part of the extract include data organized in chronological order. Accordingly, the extract is generated based on the user input and provided to healthcare data analyzer 215.

A sample HEOR analysis may include developing a research protocol, conducting a robust study design based on the research protocol, and selecting an analytical study population including several inclusion/exclusion criteria (e.g., patient age, participation in a patient support program, etc.). After identifying the study population, an exposure cohort (e.g., patients participating in a patient support program) and a no exposure cohort (e.g., patients not participating in the patient support program) are created. These cohorts are created by defining and generating appropriate extracts from central data hub 502. Outcome variables and covariates are also defined for the analysis, and a multivariable analysis is conducted (e.g., using healthcare data analyzer 215) to compare outcomes between the exposure and no exposure cohorts. The results of the analysis are interpreted, and may be published and presented to the scientific/medical community.

For a sample MABI analysis, analysis objectives are established and patient eligibility criteria are applied to eliminate sample and data capture bias. The patient eligibility criteria may be used to identify a study population. After identifying the study population, an exposure cohort (e.g., patients participating in a patient support program) and a no exposure cohort (e.g., patients not participating in the patient support program) are created. These cohorts are created by defining and generating appropriate extracts from central data hub 502. Outcome variables and covariates are also defined for the analysis, and a multivariable analysis is conducted (e.g., using healthcare data analyzer 215) to compare outcomes between the exposure and no exposure cohorts. The results of the analysis are interpreted, and a communication plan is developed and may be provided to a management team.

Those of skill in the art will appreciate that many different analyses may be performed on generated longitudinal data profiles. For example, analyses may be performed to compare different PBMs, different payers, different specialty pharmacies, etc.) Further, different patient support programs may be analyzed to determine their efficacy/value. Analysis may also be performed to compare different actors within the same patient support program. For example, if a patient support program includes a human ambassador interacting with a patient, an analysis may identify, based on patient condition, the most effective human ambassadors participating in the program. The behaviors applied by the identified ambassadors may then be communicated to the other ambassadors. In other example, an analysis may be performed as part of a customer relationship management (CRM) campaign. Extracts from central data hub 502 may also be compared with extracts from other databases in system 500. For example, extracts from central data hub 502 may be compared with extracts of identified data from marketing database 520.

In the example embodiment, ecosystem database 570, marketing database 520, and/or pharmacy database 530 may generate and transmit notifications to a HCP, patient, or third party service provider (e.g., claims data sources 504, services data sources 506, and pharmacy data sources 508) upon occurrence of a predetermined event. That is, occurrence of the event triggers generation and transmission of a notification of the event to the appropriate part. Events may include, but are not limited to, a change in patient insurance information, a change in patient address, a prescription shipment, a prescription fill, a prescription transfer from a grace filling pharmacy to a mandated pharmacy, a prior authorization expiration within a predetermined period of time, an expiry of prescription refills within a predetermined period of time, a patient failure to obtain a prescription fill within a predetermined period of time, a patient hospitalization, completion of a patient training session (e.g., training on how to administer a newly prescribed medication), any problem associated with a particular batch of a medication, a change of physician for the patient, a change in patient insurance benefits, and a patient request to replace a product related to administration of the prescription product.

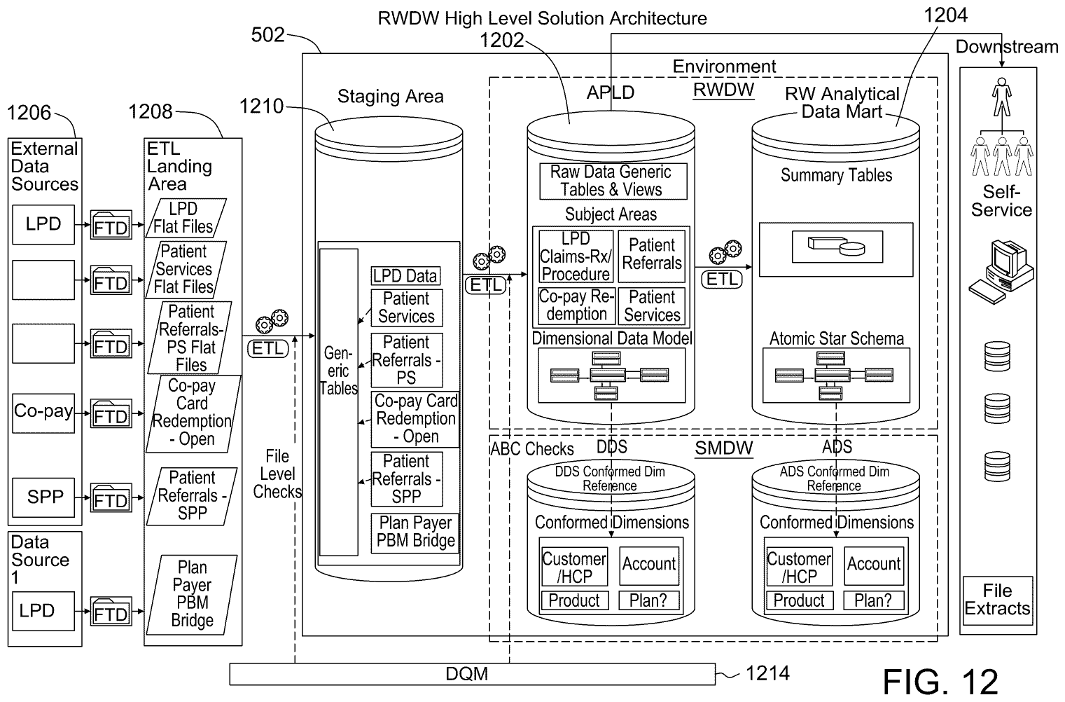

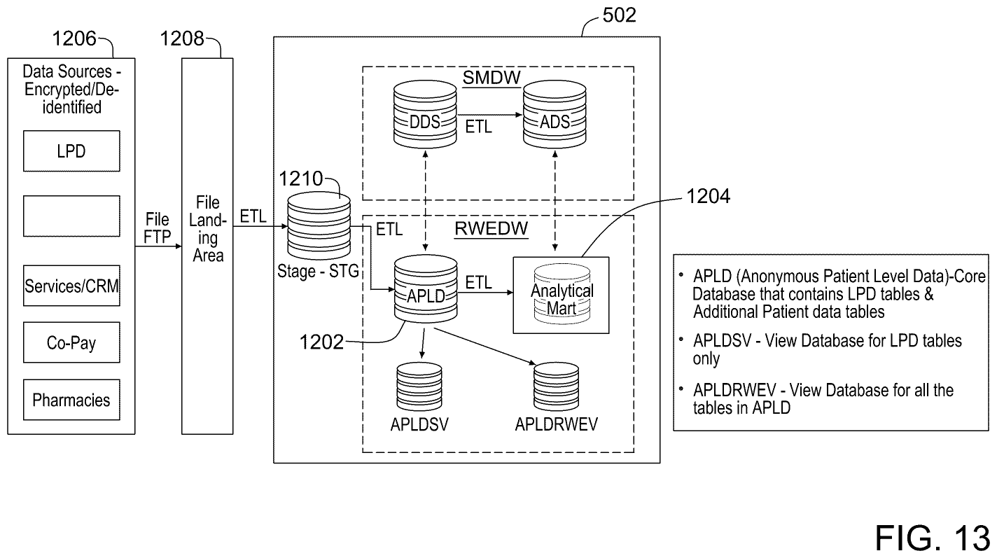

FIGS. 12 and 13 are diagrams illustrating example embodiments of an architecture of central data hub 502. In the example embodiment, central data hub 502 includes i) a detailed/integrated data layer referred to as APLD 1202 and ii) an analytical layer 1204 for data consumption for health economics outcomes research (HEOR) and marketing analytics business intelligence (MABI) studies/research. Data from various data sources 1206 is transmitted via FTP to a landing area 1208, and then transmitted by an extract, transform, load (ETL) process into a staging area 1210 of central data hub 502. Staging area 1210 includes a generic table 1212 for receiving, for example, longitudinal patient data (LPD), patient services data, patient referral data from a benefits verifier, co-pay data, patient referral data from a pharmacy, and prescription fill data.

In the example embodiment, a leveraged data quality management (DQM) framework 1214 performs checks on data in staging area 1210 and file level checks on data in landing area 1208. Further, data security is managed by creating a consumption layer with multiple view databases based on the core APLD 1202. Central data hub 502 simplifies market onboarding for LPD data, such that one ETL code base is used for any number of markets. Central data hub 502 also includes foreign key references to data distribution service (DDS) conformed dimensions such as time, customer, and product. Further, pushdown optimization is leveraged for high volume fact data refreshes from staging area 1210 to APLD 1202.

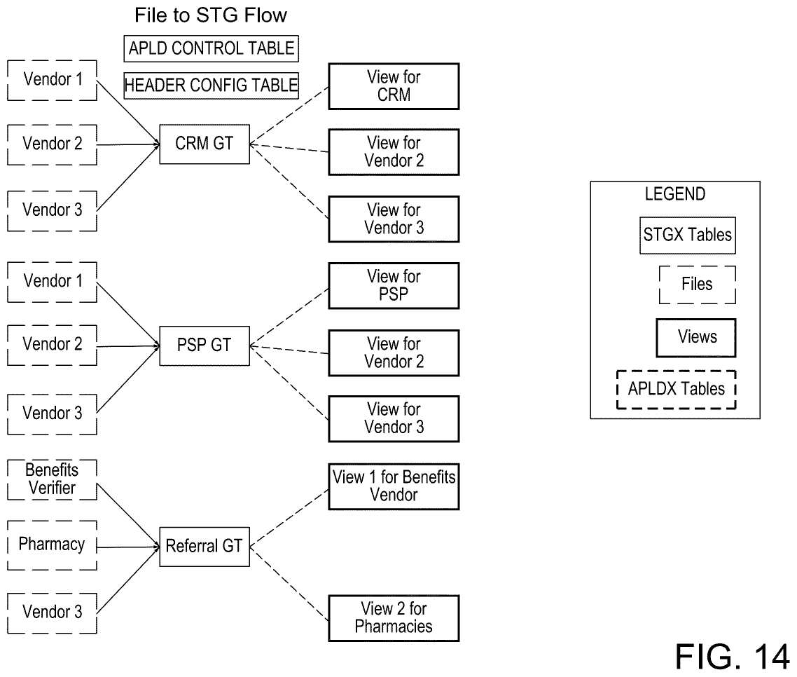

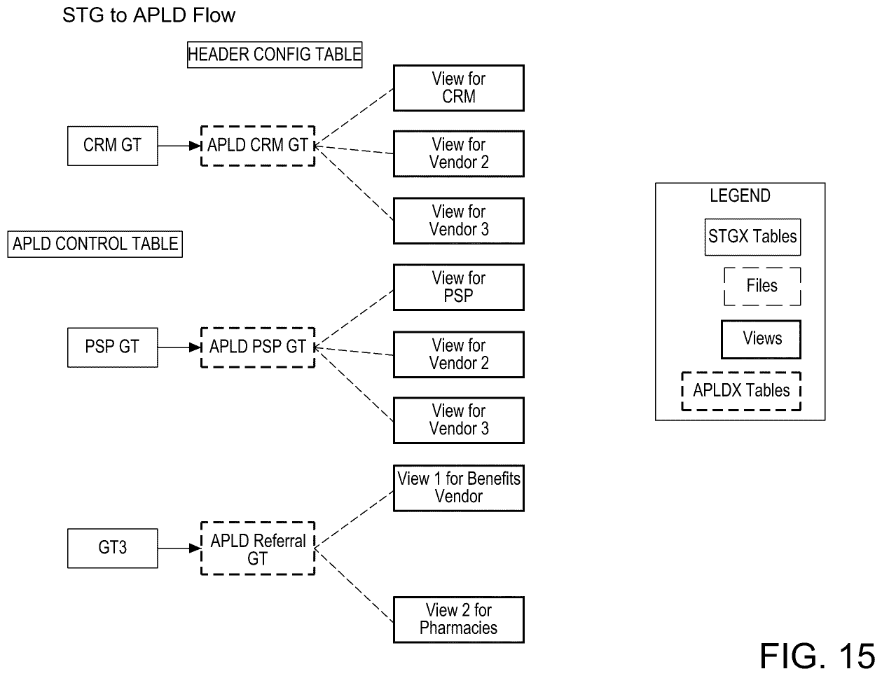

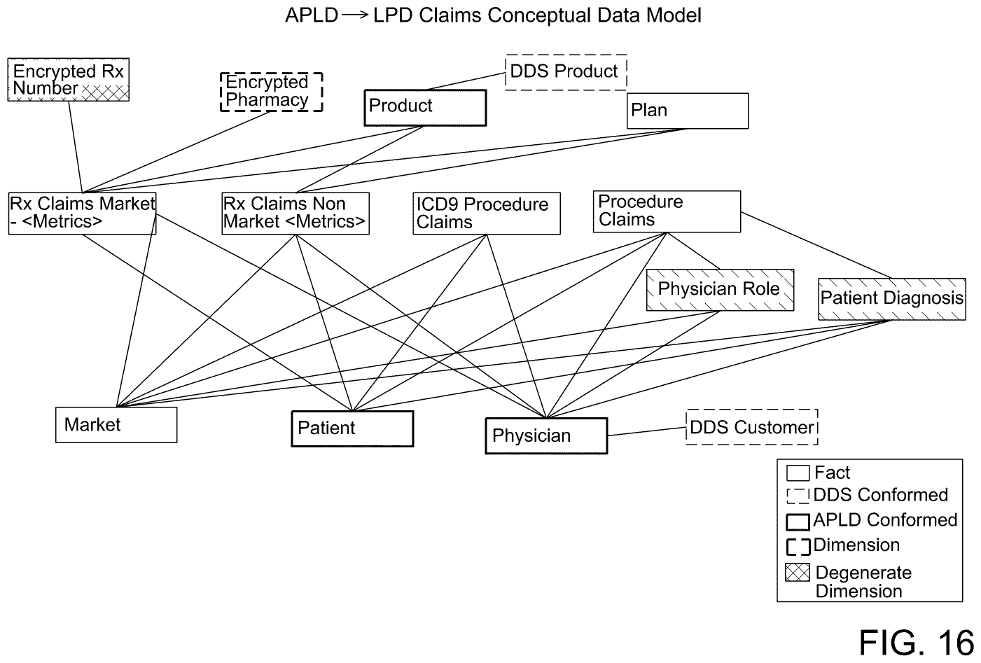

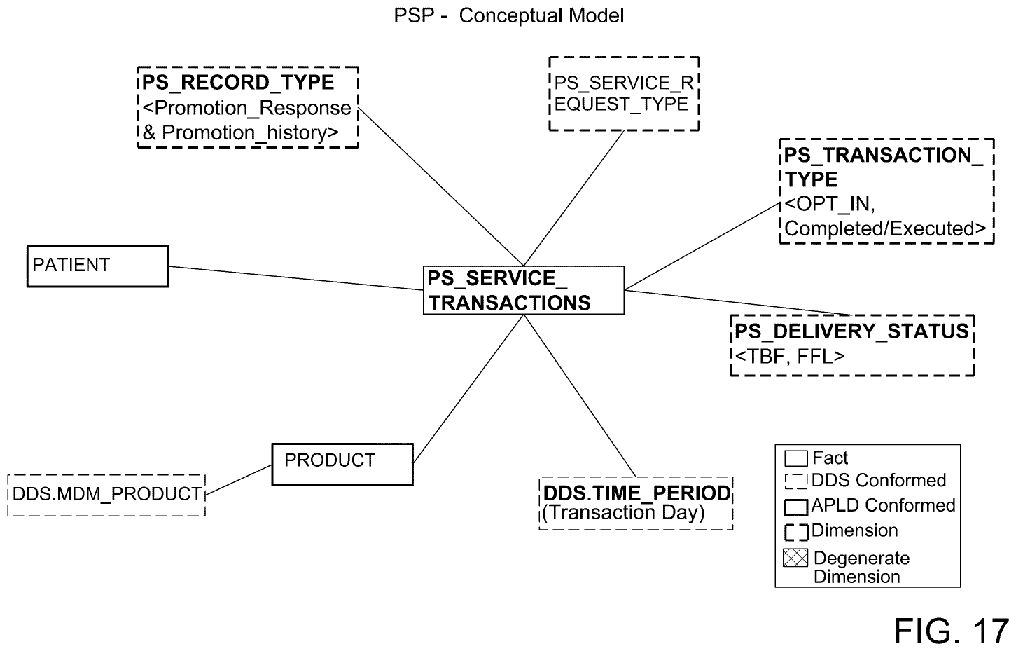

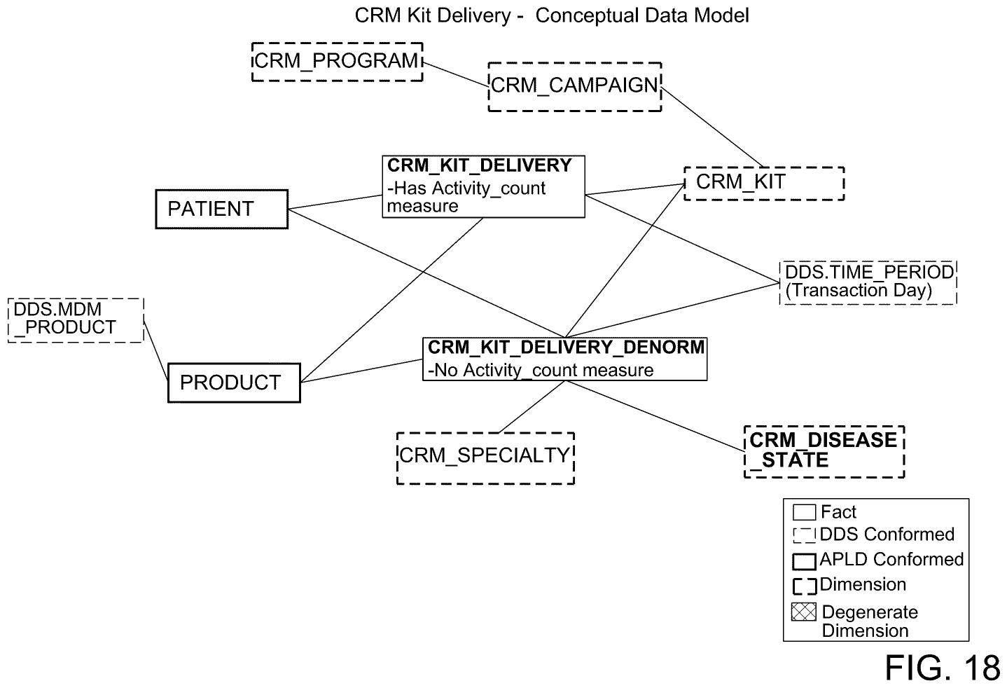

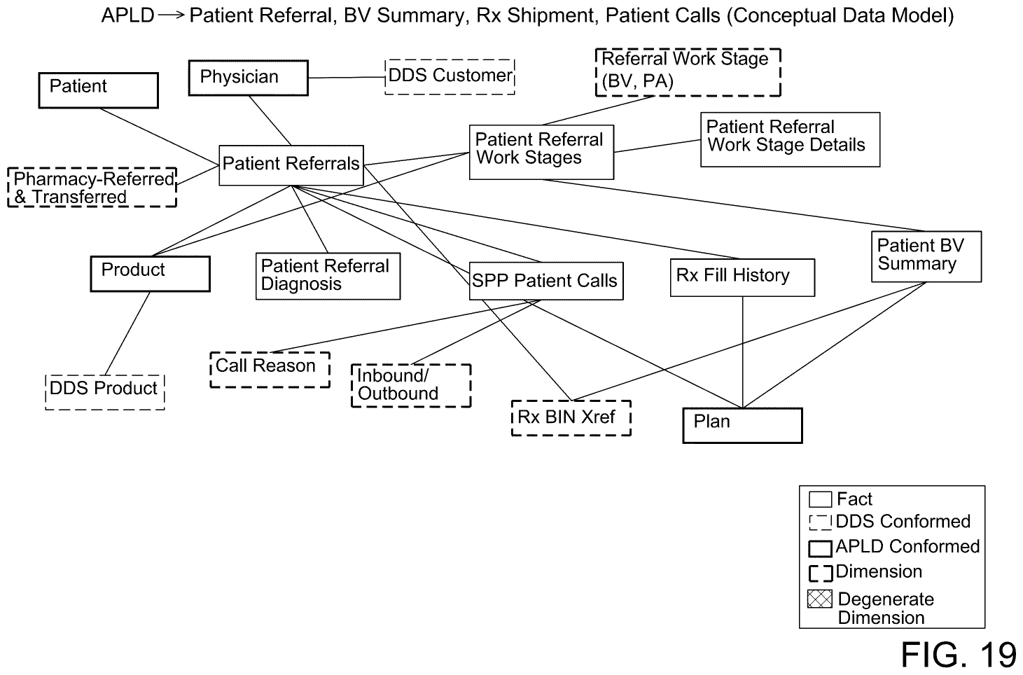

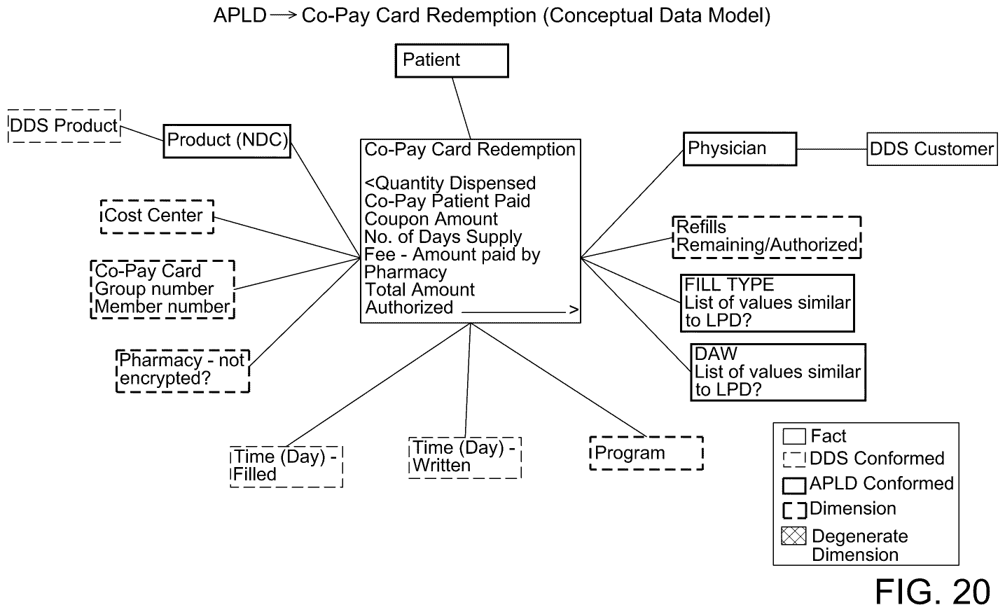

FIG. 14 is a diagram showing a flow from landing area 1208 to staging area 1210 under a generic table ETL approach. FIG. 15 is a diagram showing a flow from staging area 1210 to APLD 1202 under a generic table ETL approach. FIG. 16 is a diagram showing an LPD claims conceptual data model for APLD 1202. FIG. 17 is a diagram showing a conceptual data model for patient support program data. FIG. 18 is a diagram showing a conceptual data model for CRM kit delivery data. FIG. 19 is a diagram showing a patient referral, benefits summary, prescription shipment, and patient calls conceptual data model for APLD 1202. FIG. 20 is a diagram showing a co-pay card redemption conceptual data model for APLD 1202.

As described above, service providers 552 (shown in FIG. 5) may include a data consolidator. FIG. 21 illustrates an example data flow from a data consolidator 1402 to various other components of system 500. In the example embodiment, data consolidator 1402 is a data platform that receives benefits data (e.g., prescription benefits data and/or medical benefits data) and medication history (e.g., fill history) data. This data may be received from pharmacy benefits managers (PBMs) 1404 and/or other data sources 1406 (e.g., claims switch 702 (shown in FIGS. 7A and 7B)). Data consolidator 1402 may be a comprehensive report platform that provides a range of services, including a secure, reliable, scalable, and HIPAA compliant reports system.

Data consolidator 1402 connects to payers and data aggregators (e.g., data sources 1406) to deliver pharmacy eligibility information, pharmacy benefits information, medical benefits information, and medication history information for patients. Data consolidator 1402 may also deliver PA claims status data (e.g., data indicating whether a prior authorization form is required and/or has already been obtained for a prescription product). For example, an existing patient may have a completed prior authorization form on file, and a new prior authorization form may not be required for prescription refills. In the example embodiment, data consolidator 1402 translates raw data from a source into structured XML data, and delivers the structured XML data to various applications via service hub 550. In the example embodiment, the benefits data and medication history data is transmitted to service hub 550 in an XML format. Alternatively, the benefits data and medication history data may be provided in any suitable format.

Using the benefits data and medication history data for a particular patient, service hub 550 may, for example, calculate a drug cost for the patient and determine a co-pay card eligibility for the patient, as described in detail below. The calculated drug cost and/or co-pay card eligibility may be generated in response to a request from a potential patient made via pre-check application 568 or a request from an HCP made via HCP portal 566. In either case, the request is a web-based request transmitted from a remote user computing device to service hub 550 over the Internet.

In at least some known healthcare systems, a pharmacist may be able to obtain a calculated drug cost or co-pay card eligibility determination. However, in such systems, the pharmacist must run an actual claim, including prescription insurance information, to obtain the calculated drug cost or co-pay card eligibility determination. Further, the pharmacist may also need to provide a valid prescription to obtain the information. In contrast, service hub 550 allows a user (e.g., a patient or HCP) to directly obtain a calculated drug cost and/or co-pay card eligibility. That is, the patient or HCP, not a pharmacist, submits the request to service hub 550. Further, the patient or HCP does not need to run a claim to obtain the information, does not need to provide prescription insurance information in the request, and does not need to provide a valid prescription in the request. Rather the request may only include patient identification information (e.g., patient name, date of birth, zip code, etc.). As such, unlike at least some known pharmacy systems, service hub 550 provides a calculated drug cost and/or co-pay card eligibility directly to a user without requiring prescription insurance or valid prescription information, and without running an actual claim.

In some embodiments, the calculated drug cost and/or co-pay card eligibility may be generated in response to a user request generated using patient application 562. Further, service hub 550 may transform the XML format data into a PDF format. In some embodiments, the medication history data received from data consolidator 1402 may be limited to relatively recent (e.g., within the last year) medication history data. Accordingly, service hub 550 may periodically (e.g., yearly) retrieve medication history data and append the retrieved medication history data to previously retrieved medication history data to generate a more complete medication history for a patient. Service hub 550 may also perform data quality checks and/or data accuracy checks on data received from data consolidator 1402 to check the data for accuracy and inconsistencies. For example, data received from data consolidator 1402 may be checked for accuracy by comparing it against data previously stored by service hub 550. In another example, data received from data consolidator 1402 may be checked to ensure it matches a desired format (e.g., ensuring an estimated drug cost value is for a predetermined time interval, such as thirty days, and not for a different time interval, such as ninety days). Further, in addition to the functions described above, service hub 550 may use machine learning techniques to compile metadata regarding coverage of a prescription product for different insurance companies. Service hub 550 may also generate medication refill reminders or assign reward points as part of a patient rewards program based on the data from data consolidator 1402.

Data generated by service hub 550 may then be provided to HCP portal 566, pre-check application 568, patient application 562, and/or enterprise pharmacy application 554 in, for example, a PDF or XML format. For example, for a particular patient and prescription product, HCP portal 566 may receive and display an estimated patient cost, a deductible, any prior authorization requirements, any pharmacy restrictions, a co-pay card eligibility determination, a benefits summary, and a medication history.

The estimated product cost may include, for example, how much the prescription product will cost per year under the patient's insurance plan, whether the cost will be reduced over time, and how much of the cost may be covered by a co-pay card. When a prior authorization is required, service hub 550 may interface with a prior authorization data source to determine any electronic prior authorization restrictions. Service hub 550 may also interface with a co-pay data source to arrange delivery of a co-pay card to the patient.

Further, for a particular patient and prescription product, pre-check application 568 may receive and display an estimated patient cost, insurance information, any prior authorization requirements, a co-pay card eligibility determination, and any pharmacy restrictions. For a particular patient and prescription product, enterprise pharmacy application 554 may receive and display a benefits summary.

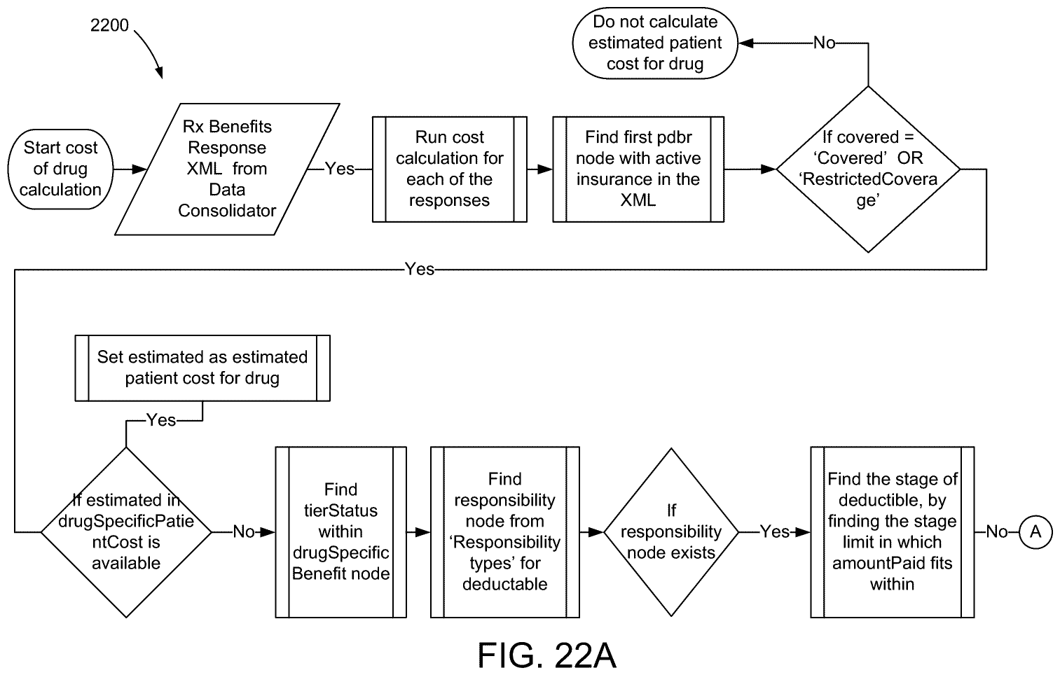

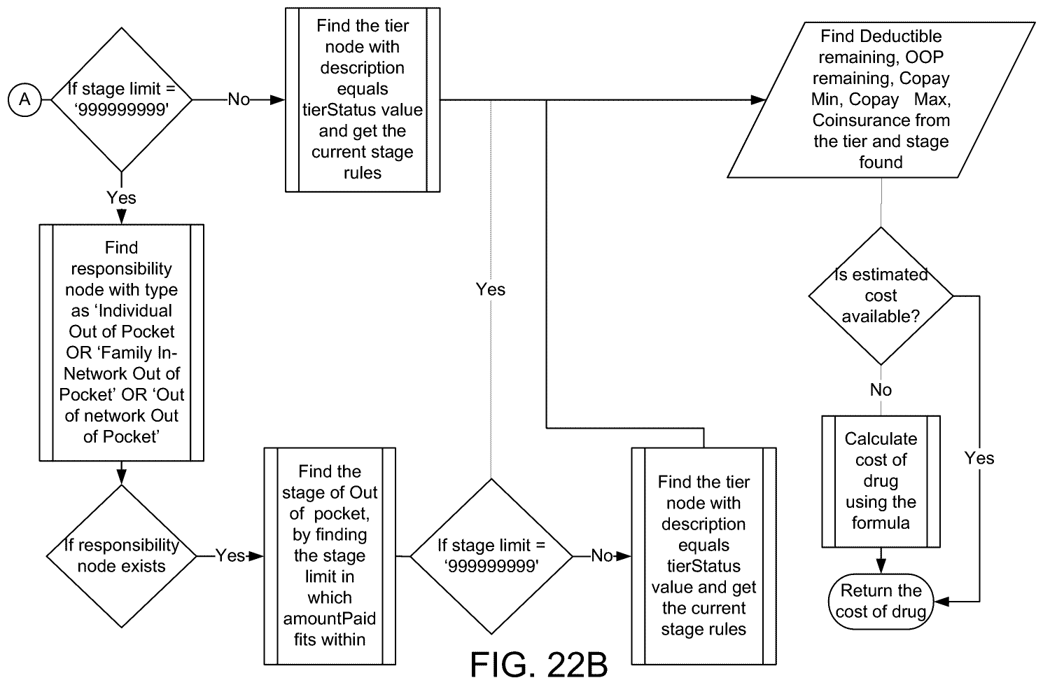

The following is an example of how an estimated patient cost for a prescription product may be calculated. FIGS. 22A and 22B show a flow diagram 2200 generally corresponding to the example process described below. Unless otherwise noted, the following calculations/determinations are performed by service hub 550. In response to a request from a user (e.g., from a potential patient or HCP), service hub 550 receives benefits data and/or a record of the patient from data consolidator 1402 and runs a cost calculation for each of the responses. If benefits data is available and a patient record has been located, service hub 550 checks a benefit period. Specifically, in the example embodiment, service hub 550 considers the first response from a PBM 1404 with currently active insurance for cost calculation.

Next, service hub 550 checks a coverage status of the patient. If the coverage status indicates the patient is not covered or ‘other’, then no estimated patient cost is returned. If the coverage status indicates the patient has coverage or restricted coverage, service hub 550 determines an applicable tier status for the patient, and determines an applicable stage for the patient. Service hub 550 checks an estimated cost of the prescription. Services hub 550 proceeds to determine whether the estimated cost of the prescription is available from the data consolidator 1402. If not, the determinations described below are performed, and the cost of the prescription for the patient is returned.

Next, service hub 550 attempts to determine a deductible amount for the patient. If a deductible node of data consolidator 1402 is not available, the deductible is set as an unknown, and service hub 550 attempts to determine whether an out of pocket (OOP) limit for the patient has been reached, or considers the estimated drug specific patient cost, when provided (e.g., in some situations, data consolidator 1402 will provide the estimated cost directly to service hub 550, and service hub 550 need not calculate the estimated patient cost). If the deductible node is available, in the example embodiment, service hub 550 checks whether the entire deductible has already been previously paid by the patient.

If the entire deductible has been previously paid, then the next stage is considered, and service hub 550 attempts to determine whether the OOP limit has been reached. For example, in a first stage, the patient may be required to pay 100% of the total estimated cost until the deductible is met. Once the deductible is met, the patient may advance to a second stage in which the patient will have to pay co-insurance of 30%.

If the entire deductible has not been paid, in the example embodiment, service hub 550 retrieves (e.g., from data consolidator 1402) a minimum co-pay amount, a maximum co-pay amount, and a co-insurance amount based on the applicable tier and stage. From the minimum co-pay amount, a maximum co-pay amount, and a co-insurance amount, service hub 550 determines which will be least expensive for the patient between co-pay and co-insurance. The least expensive option is applied towards the remaining deductible. If available, the maximum co-pay amount is applied. After this calculation, if the remaining deductible is greater than or equal to the maximum co-pay amount, then service hub 550 returns the maximum co-pay amount as the estimated drug cost. Otherwise, the co-pay amount is set equal to the remaining deductible and service hub 550 attempts to determine whether the OOP limit has been reached.

If an OOP node of data consolidator 1402 is not available, service hub 550 treats the patient's insurance plan as not having an OOP limit, and returns an OOP limit of ‘not applicable’. If the OOP node of data consolidator 1402 is available, service hub 550 checks whether an amount previously paid has reached the OOP limit. If an amount previously paid has reached the OOP limit, the estimated cost to the patient is $0.00 and no co-pay savings card needs to be applied. If the amount previously paid has not reached the OOP limit, then an OOP remaining amount is calculated as the OOP limit minus the amount previously paid. The OOP remaining amount is then applied to the drug cost.

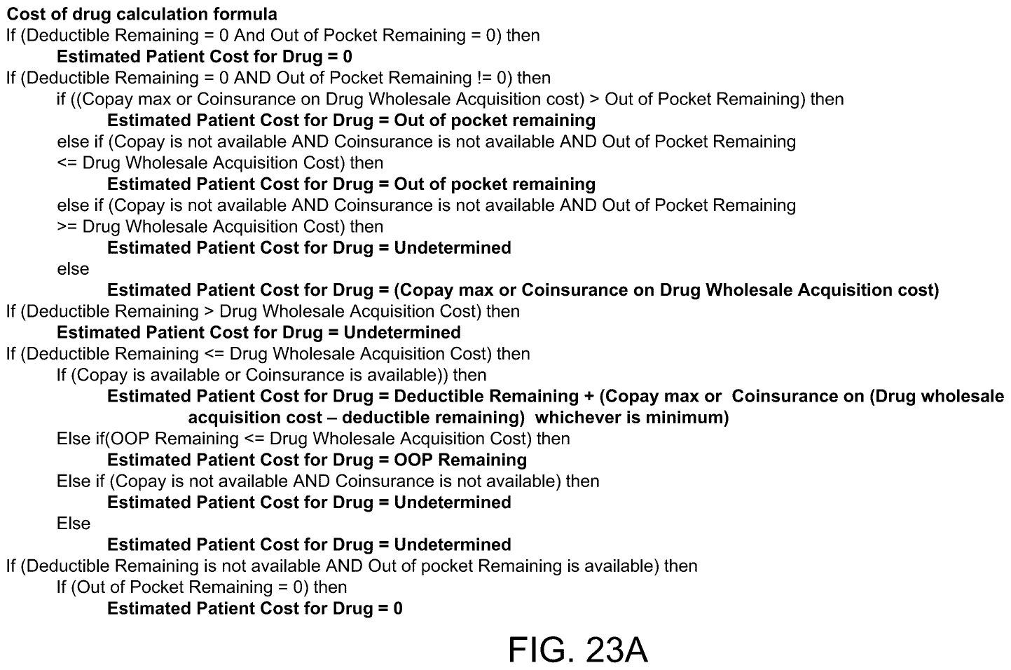

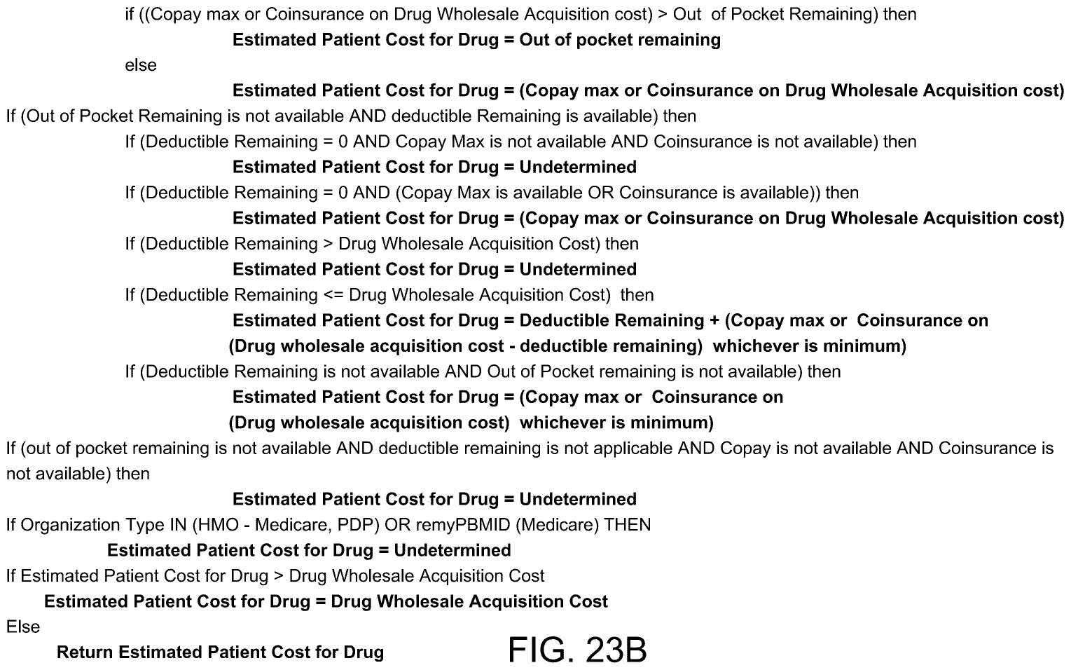

FIGS. 23A and 23B are pseudocode for an example algorithm for calculating the estimated patient cost. For illustration, a specific example of estimating cost to a patient will now be provided. Assume a patient has a deductible of $100 and an OOP of $300. Further assume that for Stage 1 the maximum co-pay amount is $80 and for Stage 2 the co-insurance is 30% of the total drug cost.

For a first fill, assume the patient is in Stage 1 and the total drug cost is $1,000. The estimated patient cost will be $180. Specifically, the estimated patient cost is calculated as the remaining deductible (e.g., $100) plus the lesser of the maximum co-pay amount (e.g., $80) and the co-insurance amount (e.g., 30% of the total cost less the applied deductible (e.g., $900)). Accordingly, the estimated patient cost is $100+$80=$180, with the remaining deductible reduced to $0 and the OOP limit reduced to $120 (e.g., $300-$180).

For a second subsequent fill, having exhausted the deductible, the patient is now in Stage 2. The total drug cost is still $1,000, and the estimated patient cost is now $80. Specifically, there is no remaining deductible, so the estimated patient cost is the lesser of the maximum co-pay amount (e.g., $80) and the co-insurance amount (e.g., 30% of the total cost (e.g., $1000)). The $80 estimated patient cost for the second fill reduces the OOP limit to $40 (e.g., $120-$80).

For a third fill, the total drug cost is still $1,000, and the estimated patient cost is now $40. Specifically, there is no remaining deductible, so the estimated patient cost is initially calculated as the lesser of the maximum co-pay amount (e.g., $80) and the co-insurance amount (e.g., 30% of the total cost (e.g., $1000)). However, at this point, the OOP limit is $40, so the actual estimated patient cost is $40. This reduces the OOP to limit to $0.

Finally, for a fourth fill, there is no remaining deductible, and the OOP limit is $0. Accordingly, the estimated patient cost for the fourth fill is $0.

The following is an example of how co-pay eligibility for a prescription product may be calculated. Unless otherwise noted, the following calculations/determinations are performed by service hub 550. In response to a request from a user (e.g., from a potential patient or HCP), service hub 550 returns a co-pay eligibility determination to the user. Service hub 550 may determine co-pay eligibility based on, for example, a prescription product, an indication for the prescription product, the patient's age, and a status of the patient's insurance plan. For example, for certain indications, the patient may need to be a certain age (e.g., 18 or older) to be eligible for a co-pay card. In another example, if the status of the patient's insurance plan cannot be determined, then service hub 550 may notify the user that the co-pay eligibility determination is incomplete.

As noted above, in the example embodiment, the estimated patient cost and the co-pay eligibility determination are returned in response to a request received from pre-check application 568 or HCP portal 566. In the example embodiment, the request includes patient identification information (e.g., patient first and last name, patient date of birth, patient gender, patient zip code). The request for the estimated patient cost and/or the co-pay eligibility determination may be forwarded to service hub 550, which communicates with ecosystem database 570 and/or service providers 552 (e.g., data consolidator 1402) to retrieve data needed to generate estimated patient cost and/or the co-pay eligibility determination. The data is retrieved based on the patient identification information supplied in the request.

In some embodiments, electronic marketing data related to the prescription product may be retrieved and analyzed to identify prospective patients. The prospective patients may be stored in a prospective patient database. Then, a patient database may be compared to the prospective patient database to identify patients. Based on that comparison, the electronic marketing data may be analyzed (e.g., to determine the impact of marketing on the prospective patients).

As described above, healthcare data processing system 500 may include a patient application (e.g., patient application 562, shown in FIG. 5). The patient application may be used in conjunction with healthcare data processing system 500, or may be used independent of healthcare data processing system 500.

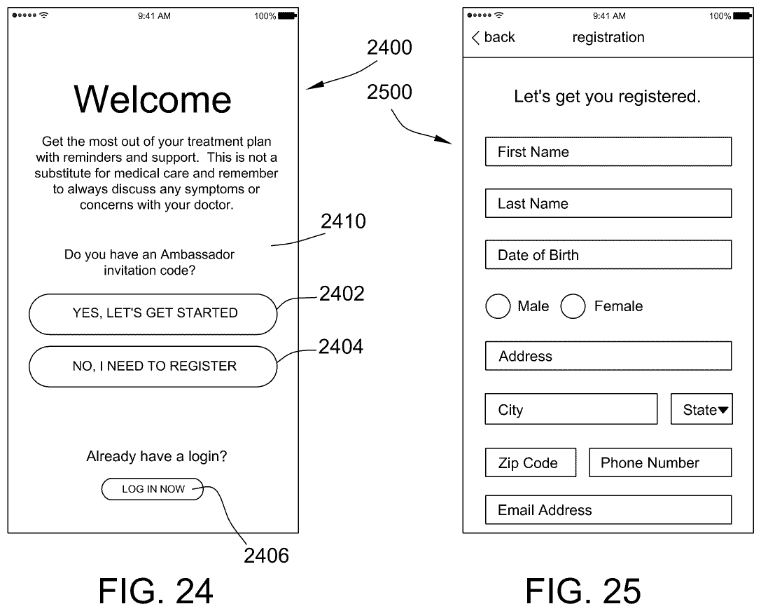

FIGS. 24-49 include screenshots of one example embodiment of the patient application as part of a coordinated care platform (e.g., coordinated care platform 560, also shown in FIG. 5). The patient application may be accessible on any suitable electronic device, such as a mobile phone, tablet, watch, or any other computing device. Additionally, the patient application may facilitate communication between the computing device and another connected device, including, for example, a fitness or health wearable, in order to incorporate other fitness or wellness tracking as part of the user's profile, and/or a connected injection device, in order to monitor medication usage or consumption data. The computing device and the connected device may be communicatively coupled, for example, via a wireless connection such as Bluetooth. The patient application enables a patient to log-in, track their medication regime, schedule injections, log symptoms and injections, and track their progress in their regime as compared to other patients using the same medication. In some embodiment, the patient application also enables the patient to order a copay savings card, order medication supplies (e.g., a sharps container), enroll in a medication ambassador program, and/or subscribe to medication reminders via email, phone, and/or text (e.g., such that the patient receives an email/phone/text reminder prior to or at the time when the medication should be administered). Using the patient application, the patient may also synchronize account data for the patient (e.g., username, password, etc.) between a first account associated with a website for the prescription product and a second account associated with the patient application. For example, if a patient initially creates an account on the website, and later downloads the patient application, the patient can, using the patient application, import their account data from the website into the patient application and the patient data may be synchronized between these various platforms/devices. In addition, the patient application provides a rewards program that allows patients to earn in-application rewards (such as new backgrounds) as well as real-world rewards (such as discounts at participating pharmacies, for example by displaying a barcode that is then scanned at the pharmacy to apply a discount to a product purchase).

The patient application may be configured to communicate with various other software and/or applications on the patient's computing device. For example, the patient application may be able to access or otherwise communicate with other health-related applications, including other fitness, health, and/or medication tracking applications or health kit applications. The patient application may be configured to retrieve data from and/or report data to these other applications. In addition, the patient application may be configured to track, monitor, and/or record application utilization metrics for the patient, such as how often the patient accesses the patient application, and the various features of the patient application used by the patient.

In one embodiment, the patient application, once downloaded onto the patient's computing device, may not require internet connectivity to perform some or all of the functionality of the patient application (e.g., setting alerts, notifying the patient with those alerts, tracking the patient's treatment regimen, tracking symptoms). In some embodiments, all or a portion of the data input by the patient into the patient application (including, for example, application utilization metrics, medication consumption, symptom logs, and/or injection logs) may be electronically transmitted to a central data hub (e.g., central data hub 502) for processing, and the processed data may be transmitted for further processing and/or display by the patient application.

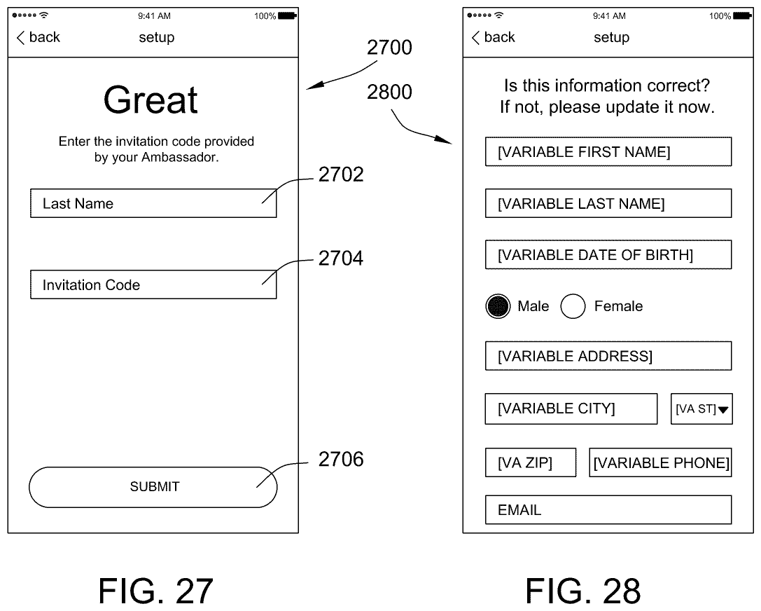

FIG. 24 illustrates a welcome screen 2400 of the patient application. In the example embodiment, the welcome screen 2400 provides a patient with three options, although any suitable number of options may be provided in alternate embodiments. If the patient has received an invitation code to the patient application, for example, from a medication ambassador, the patient may select a first option 2402. As used herein, a “medication ambassador” refers generally to healthcare personnel that are specially trained to provide guidance, training, support, and/or advice regarding a medication. Selecting the first option 2402 takes the patient to the account set-up page 2700 illustrated in FIG. 27. If the patient has not received an invitation code, the patient may select a second option 2404, indicating that the patient needs to register with the patient application. Selecting the second option 2404 takes the patient to the registration page 2500 illustrated in FIG. 25. If the patient has already been through the registration process (e.g., this is not their first time opening or interacting with the patient application), the patient may select a third option 2406 to log in. Selecting the third, log-in option 2406 takes the patient to a log-in screen (not shown) where the patient may enter their email address and PIN. If the patient has already logged in on their computing device (e.g., client system 204, shown in FIG. 1), their email address may be pre-populated into an email field to save the patient the time and effort of filling in the field.

In the example embodiment, a background image 2410 on the welcome screen 2400 and registration and set-up screens (shown in FIGS. 25-34) is blurred. After the patient completes a successful log-in, the image 2410 clears. In some embodiments, other portions of the welcome screen 2400 or additional screens (not shown) may be distorted or de-activated (e.g., grayed-out) until the patient's data has been verified. In addition, the background image 2410 is randomized and will refresh or change with each new session (e.g., each time the patient opens the patient application anew). The number of images in rotation depends on the number of images the patient has “earned” using in-app rewards, as will be described more fully herein.

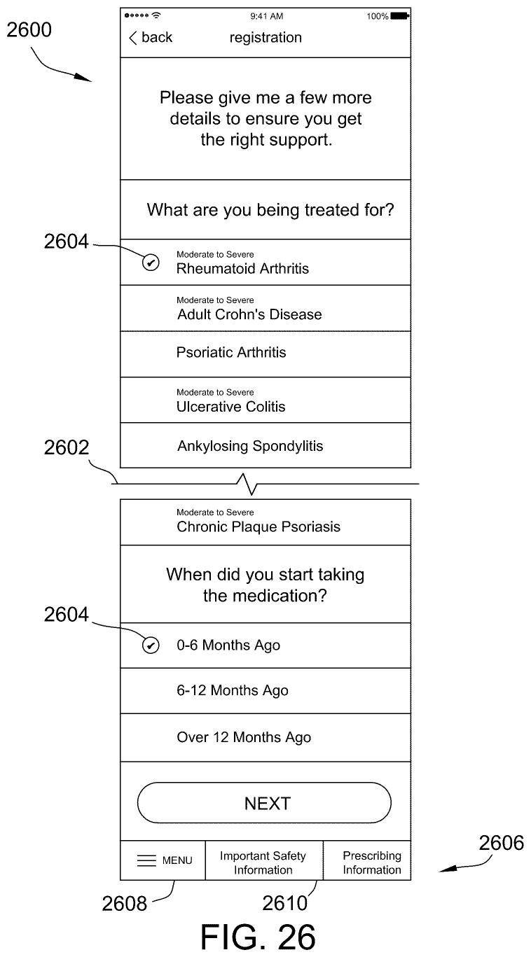

FIG. 25 illustrates an initial registration page 2500 of the patient application. All of the variable fields are blank, indicating that the patient may be required to manually fill in all fields to complete the registration process. Upon completion of the initial registration page 2500, the patient may select a “save and continue” option (not shown) to move on to the next step in the registration process, shown in FIG. 26.

FIG. 26 illustrates another registration page 2600 of the patient application. It should be understood that the patient may have to scroll down or otherwise navigate a user interface of the patient application to view all of the information shown, as indicated by page break 2602. In the example embodiment, the registration page 2600 prompts the patient to select which condition(s) they are being treated for (e.g., why they are using the medication) and when they began taking the medication. A checkmark or other selection symbol 2604 will appear next to the condition and time selected by the patient. The patient may only be able to select a single condition.

In addition, a footer 2606 is present on the registration page 2600. A “menu” icon 2608 is grayed-out or deactivated, because the menu features may only be available after the registration process has been completed. An “Important Safety Information” (ISI) icon is active. During the registration process, the user may be required to review the ISI in totality and acknowledge the information contained therein in order to continue to the next step of registration. The ISI for the associated medication may then be available to read at any point in the registration process. The ISI includes, for example, warnings, side effects, and instructions associated with the medication.

Although not shown, the patient may also be required to read and/or acknowledge terms of use and/or a privacy policy associated with the patient application during the registration process. The terms of use and/or the privacy policy may indicate to the patient that their information may be collected and anonymized and/or aggregated in order to compare the patient to various other patients using the medication (e.g., showing the patient's progress or compliance compared to other, similar patients) and/or to monitor compliance data for one or many patients. In situations in which the patient application discussed herein collects personal information about patients, or may make use of personal information, the patients may be provided with an opportunity to control whether programs or features collect patient information (e.g., “opt out” of data collection). In one embodiment, collected data may be identifiable to the patient and accordingly may be collected and/or stored securely, for example, in an encrypted format. In another embodiment, certain data (such as compliance or adherence data) may be treated in one or more ways before it is stored or used, such that personally identifiable information is removed. For example, a patient's identity may be treated so that no personally identifiable information can be determined for the patient, and/or a patient's data may be aggregated and/or anonymized such that no information may be personally associated with the patient. Thus, the patient may have control over how information is collected about the patient and used by patient application.

FIG. 27 illustrates an initial account setup page 2700 for a patient with an invitation code. The page 2700 includes a field 2702 for the patient to enter their last name, and a field 2704 for the patient to enter their unique invitation code. Upon completion of the fields 2702, 2704, the patient may select a “submit” option 2706. The patient application will verify the invitation code. If the invitation code is valid, the setup page 2800 illustrated in FIG. 28 will be displayed.

FIG. 28 illustrates a setup verification page 2800 for a patient with a valid invitation code. In contrast to the registration page 2500 shown in FIG. 25, the fields in the setup verification page 2800 are pre-populated for the patient based on information input by the HCP or medication ambassador that issued the invitation code to the patient. Each variable in the field is pre-populated according to the patient's unique information. The patient is able to change any incorrect information and, upon completion of the setup verification page 2800, may select a “save and continue” option (not shown) to proceed.

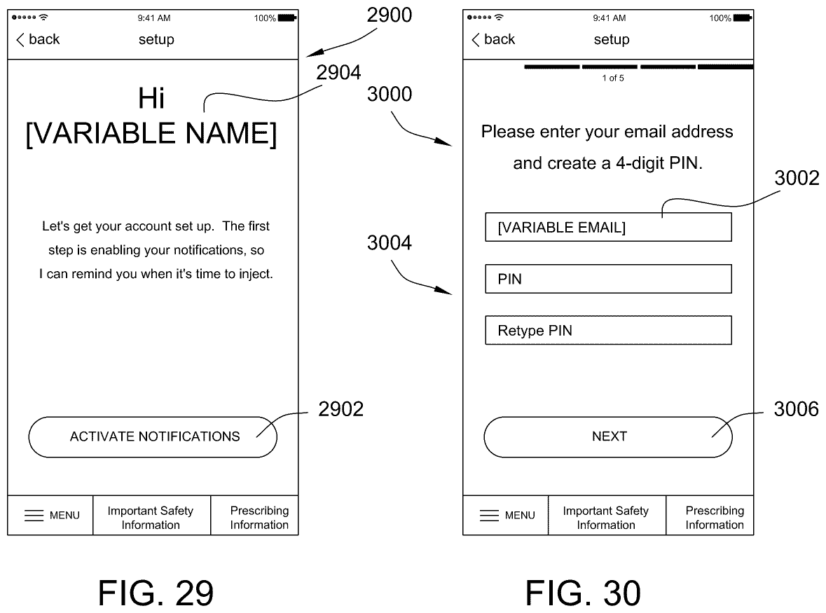

FIG. 29 illustrates a notification setup screen 2900. The patient may select an “activate notifications” option 2902, which will cause the display of a dialog requesting permission from the patient for the patient application to display notifications. The dialog (not shown) is specific to the computing device on which the patient is accessing the patient application. A “variable name” field 2904 is populated with the patient's name, as entered in the registration page 2500 in FIG. 25 or the setup verification page 2800 in FIG. 28.

FIG. 30 illustrates a first set-up page 3000 including PIN set-up. A “variable email” field 3002 is populated with the patient's email, as entered in the registration page 2500 in FIG. 25 or the setup verification page 2800 in FIG. 28. The patient is prompted to enter a PIN into two PIN fields 3004. The PIN acts as a unique security feature, like a password, that the patient will enter during future log-in to the patient application (along with the patient's email address), such that their data within the patient application is secure. Upon selecting a “next” option 3006, the patient may be presented with a confirmation screen (not shown) indicating the PIN set-up was successful.

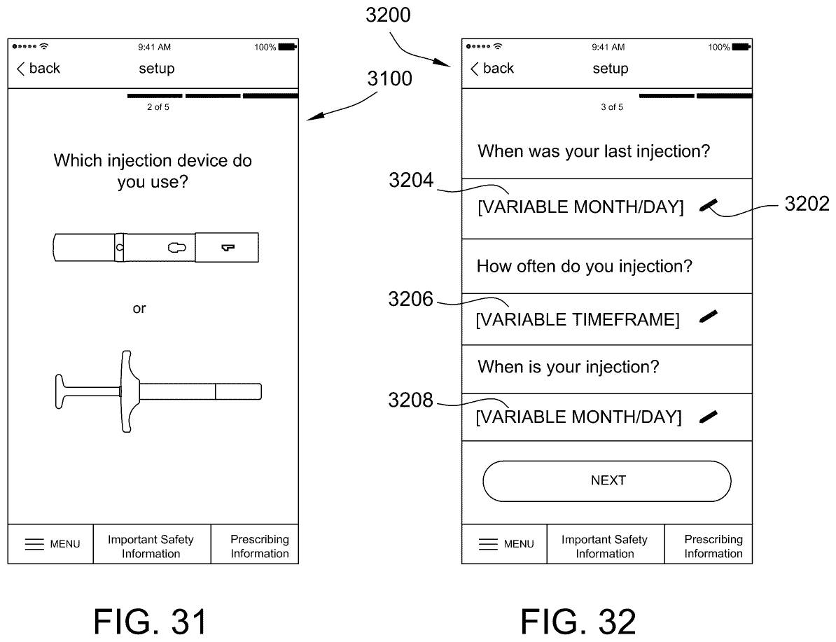

FIG. 31 illustrates a second set-up page 3100 including device selection. In the example embodiment, the patient is prompted to select which injection device, of a pen or a syringe, they use to administer their medication. In other embodiments, there may be fewer or more device options or alternate delivery options, based on the medication (e.g., orally via a pill or capsule, via a patch worn on the skin, a pump, etc.).

FIG. 32 illustrates a third set-up page 3200 including a schedule set-up. Each field includes a corresponding icon 3202, shown as a pencil in the example embodiment, indicating the patient may manipulate the value(s) in the field. In the example embodiment, the patient selects each field by tapping or selecting anywhere within the field, and chooses an appropriate response to manually set up the schedule for their treatment regimen, as prescribed by their HCP. In another embodiment, the patient application may be configured to receive the patient's schedule for their treatment regimen and pre-populate the scheduling fields according to a prescription from the patient's HCP and/or the patient's medication ambassador.

A first field 3204 prompts the patient to enter the date of their last dosage (e.g., injection). In the example embodiment, the patient enters the date in a month/day/year format, or may select an option indicating that the patient has not yet taken a first dosage (e.g., has not injected yet). A second field 3206 prompts the patient to enter how often they have been directed by their HCP to take another dosage of their medication (e.g., how often they inject). In the example embodiment, there are two intervals options including “every week” and “every other week.” In other embodiments, there may be other interval options, including, for example, “every day,” “every other day,” and/or “every month.” A third field 3208 prompts the patient to enter the date of their next dosage (e.g., their next injection). In the example embodiment, the patient may enter a date up to two weeks into the future from the set-up date. In other embodiments, additional date options may be available, for example, up to a month in the future from the set-up date. In some embodiments, all or some of the data input by the patient may be automatically electronically retrieved based on data previously input by the patient and/or may be saved on a central data hub (e.g., central data hub 502).

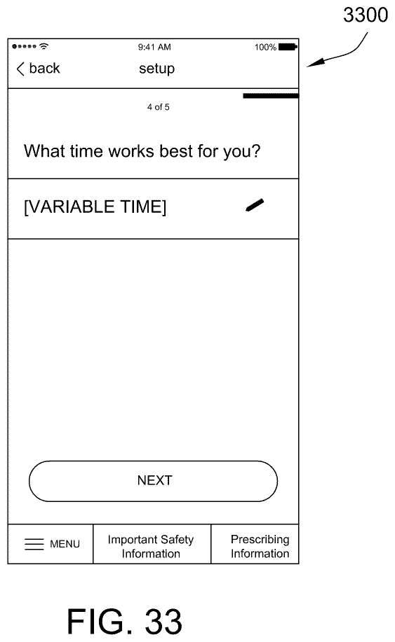

FIG. 33 illustrates a fourth set-up page 3300 including a timing set-up. The patient is prompted to enter a time of the day at which it is best for the patient to take the dose of their medication. The patient may enter any hour/minute combinations from 12:00 am to 11:45 μm, in 15-minute increments.

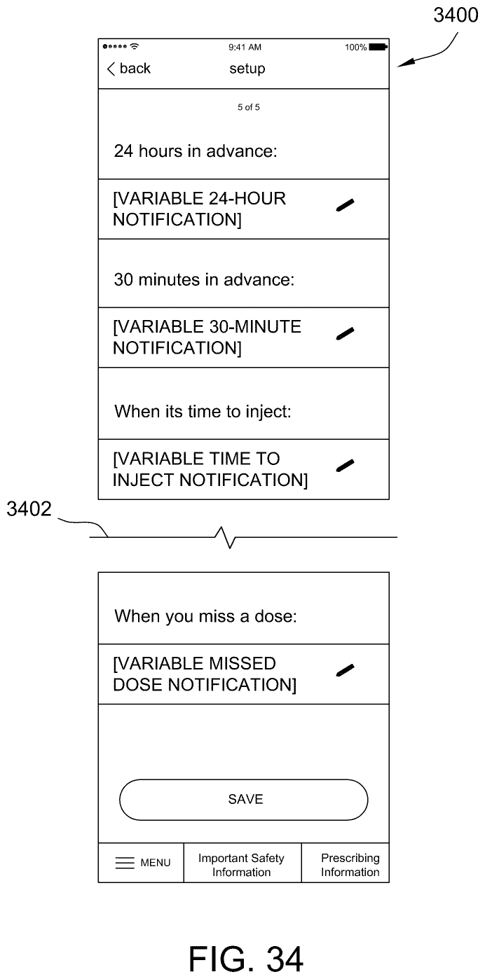

FIG. 34 illustrates a fifth set-up page 3400 including a notification set-up. It should be understood that the patient may have to scroll down or otherwise navigate a user interface of the patient application to view all of the information shown, as indicated by page break 3402. The patient may select which notification(s) to receive in certain situations associated with taking a dose of (e.g., injecting) their medication. In the example embodiment, the patient may select a notification for 24 hours and 30 minutes before their dose, a time to inject, and when the patient misses a dose. Each notification is configured to include the patient's name populated in “Variable Name.” For injections that the patient may self-perform, notifications may be configured to reference the patient's next dose or injection (e.g., “Don't forget, [Variable Name], your next dose is tomorrow”). For injections that the patient may be required to visit an HCP to receive, the notifications may be configured to reference the patient's appointment to receive their injection (e.g., “Don't forget, [Variable Name], your appointment is tomorrow.). Missed-dose reminders may prompt the patient to update their compliance log and/or to adjust their medication schedule (e.g., “Has your dosing schedule change, [Variable Name]? You may need to update your log or adjust your reminders.”) The notifications set-up on the fifth set-up page 3400 will be displayed on the patient's computing device according to the device setting (e.g., as a push notification or as a message on a device lock screen).

The patient may also enable or activate periodic reminders. Periodic reminders may be displayed on the patient's computing device to prompt the patient to log their symptoms. The periodic reminders may randomize or cycle through various stock messages (e.g., “How are you feeling? Take a moment to update your log” and “Got a moment? Log how you've been feeling”) to minimize “notification-blindness,” or a de-sensitization of the patient to notifications from the patient application. Periodic reminders may be displayed at certain interval(s), such as once a week. In some embodiments, the patient may be able to set up additional notifications, including medication refill reminders; medication shipping and/or delivery notifications; and/or notifications for the availability of new educational, safety, or training information.

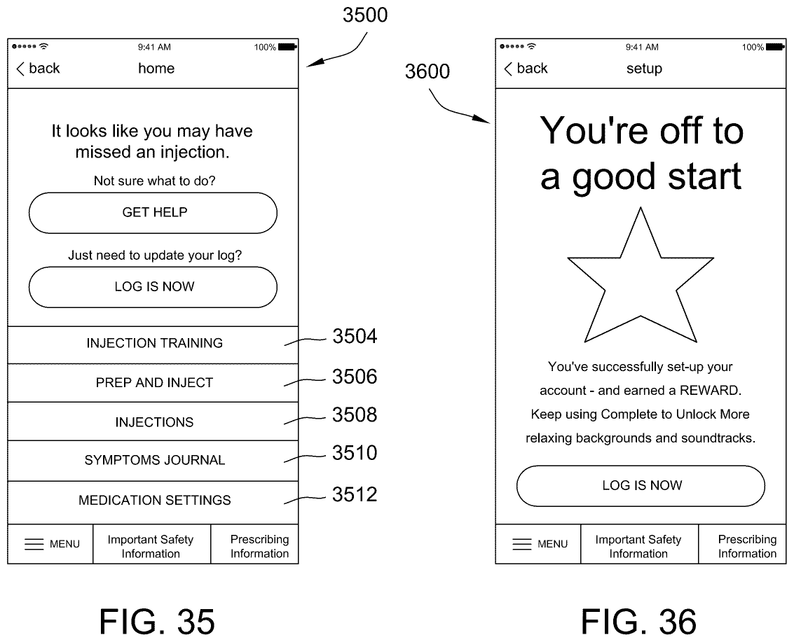

FIG. 35 illustrates a home screen 3500 including a missed-dose notification 3502. The home screen 3500 with the missed-dose notification 3502 may be displayed when the patient misses a dose. The patient may select an option to contact their medication ambassador and/or their HCP in the event the dose has been missed. If the patient has merely forgotten to log their dose, the patient may select an option to log the dose.

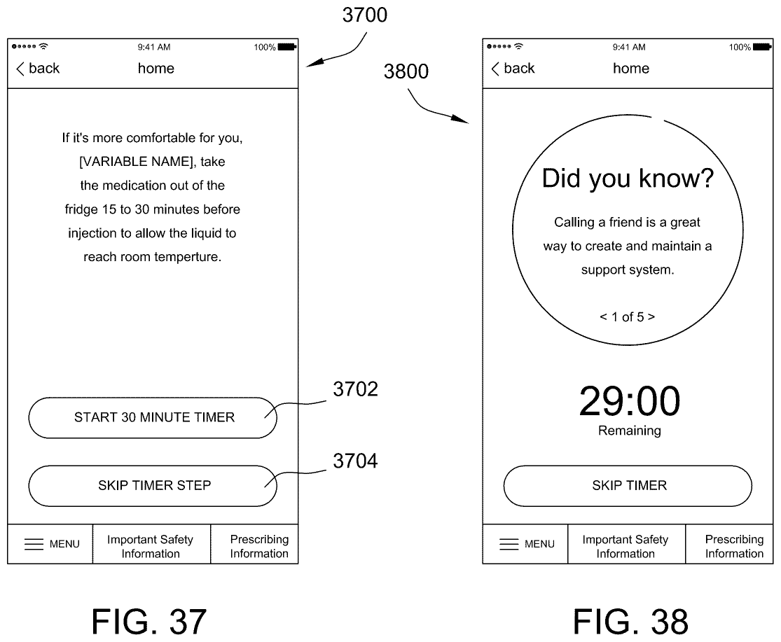

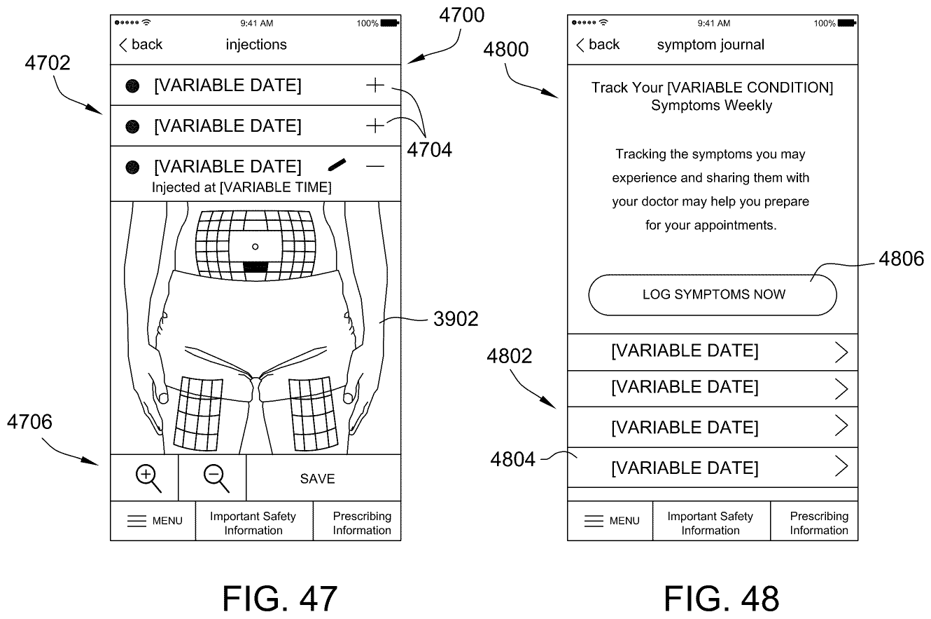

Other embodiments of the home screen 3500 may include messages or notifications for the patient, including messages of encouragement (e.g., “You're on the right track, [Variable Name]. Keep it up!”) or reminders (e.g., “Your next dose is in 5 days” or “Your next dose is tomorrow”). The home screen 3500 also includes navigation options for the patient. An “injection training” option 3504 enables the patient to view tutorial video(s) and other educational materials regarding their medication. A “prep and inject” option 3506 enables the patient to initiate an injection preparation process, described with respect to FIGS. 37-45. An “injections” option 3508 enables the patient to view various details about their past injections, including injection dates and sites, described with respect to FIGS. 46 and 47. A “symptoms journal” option 3510 allows the patient to log their symptoms and/or to view past symptom logs. A “medication settings” option 3512 allows the patient to view and/or update their medication settings, including their medication schedule and notification settings, input during the set-up phase of the patient application.

FIG. 36 illustrates a rewards screen 3600. In the example embodiment, the rewards screen 3600 is displayed when a patient earns rewards. The patient may earn rewards after the patient completes certain tasks within the patient application, including completing registration and set-up, logging a symptom, and completing a satisfaction survey (e.g., taking a brief survey about their experience with the patient application). Creating an injection log (e.g., adhering to a treatment regimen) may also result in a reward (one per each log created), but the patient may not be able to claim the reward until the start of the patient's next session (e.g., the next dose). Additionally or alternatively, the patient may claim a reward for logging into and/or using the patient application (e.g., at regular intervals or a certain number of times). As described more fully herein, the patient application includes soundtrack options that play while a patient takes their medication dose (e.g., injects) to relax and/or focus the patient. In the example embodiment, in-application rewards include additional background images and additional soundtrack options.

The rewards earned in the patient application may also be redeemable outside of the application. For example, certain rewards may be redeemable for discounts, offers, coupons, or products at participating pharmacies or other merchants (e.g., rewards may be displayed in the form of a scannable barcode for redemption at a merchant location). In addition, the patient may be rewarded for compliance or adherence to their treatment regimen, or for how many in-app rewards they have earned compared to other users of the patient application. Additionally or alternatively, the patient may be rewarded for having an application utilization that is higher (e.g., in percentage), relative to other users of the application (e.g., if the patient's application usage is in the top 5% or 10% of users of a similar age, area, or condition). The patient may be rewarded, as described above, for logging their doses and complying with their regimen. The patient may also be rewarded for having a level of compliance or adherence that is better than other users of the medication (e.g., if the patient's compliance is within the top 5% or 10% of users of a similar age, in a similar area, or taking the medication to treat the same condition). More specifically, in at least some implementations, the patient application displays the standing of the patient as compared to other patients in an anonymized manner. In some implementations, the standing patient application displays the standing of the patient (e.g., percentile) as compared to an anonymous set of patients having one or more selected characteristics in common with the patient (e.g., age range, geographic region, medical condition).

FIG. 37 illustrates a first injection preparation page 3700. The first injection page 3700 may be displayed if the patient responds to a notification that it is time to prepare for their injection (e.g., either a 30-minute notification or a “time to inject” notification) by opening the patient application. The patient may select a first option 3702 to begin a 30-minute timer before they inject their medication. Taking such time before an injection may relax and/or focus the patient to make the injection easier for the patient to perform. The patient may alternatively select a second option 3704 to skip the 30-minute timer to being the injection process.

FIG. 38 illustrates a second injection preparation page 3800 including a 30-minute timer. In the example embodiment, a circle 3802 steadily changes color over the course of 30 minutes, while a timer 3804 counts down in one-second increments from 30:00 to 00:00. A “Did you know?” message 3806 is shown within the circle with facts and/or helpful tips for the patient. In the example embodiment, the message 3806 is updated every six minutes. The timer 3804 defaults to a 30-minute countdown, but the patient may tap through multiple “Did you know?” messages 3806 to decrease the time left on the timer 3804. It should be understood that the second injection preparation page 3800 as shown in FIG. 38 is illustrative only, and as much may be arranged other than depicted in FIG. 38, including fewer or additional elements or a differently configured timer, without departing from the scope of the disclosure.

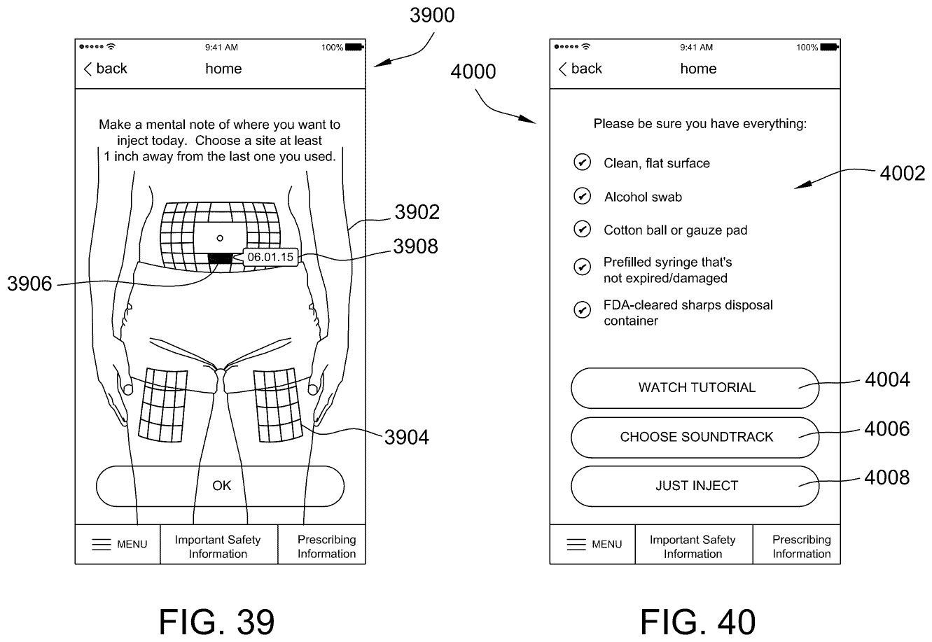

FIG. 39 illustrates a last injection screen 3900 that is displayed at the beginning of the patient's injection process. In one embodiment, the patient should inject a current dose at least one inch away from the site of a last dose. Accordingly, the patient application presents a view of the last injection site to remind the patient where they should not inject. In the example embodiment, the last injection site is displayed on a body image 3902 of a female or a male, based on the patient's specified gender (input in the registration process). White grid lines 3904 are overlaid on the body image 3902 to indicate appropriate one-inch intervals, and colored grids 3906 indicate the specific site of the last injection. The colored grid(s) 3906 may also include a date tag 3908 to identify the specific date of the injection at the respective colored grid 3906. In the example embodiment, the last injection screen 3900 may only display the site(s) 3906 associated with the last (e.g., most recent) date of injection. Based on the patient's dosage, up to four sites 3906 may be associated with the last date of injection.

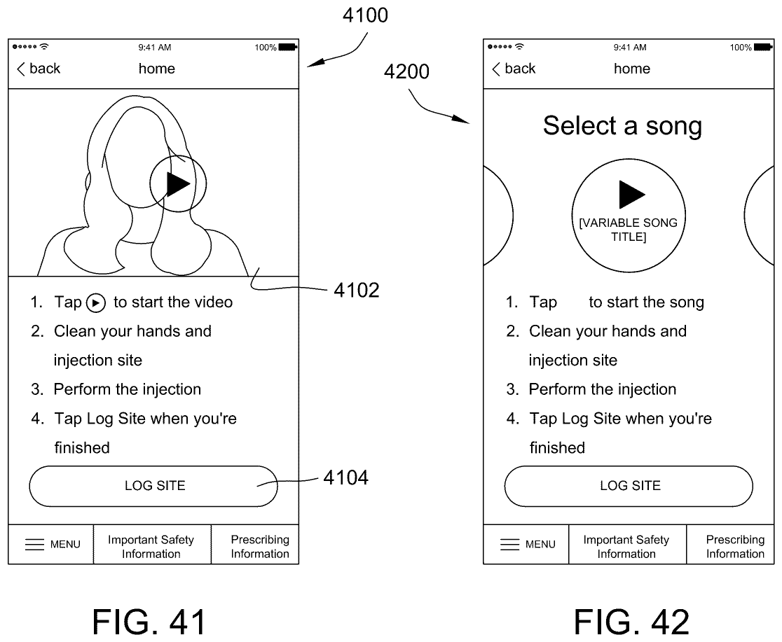

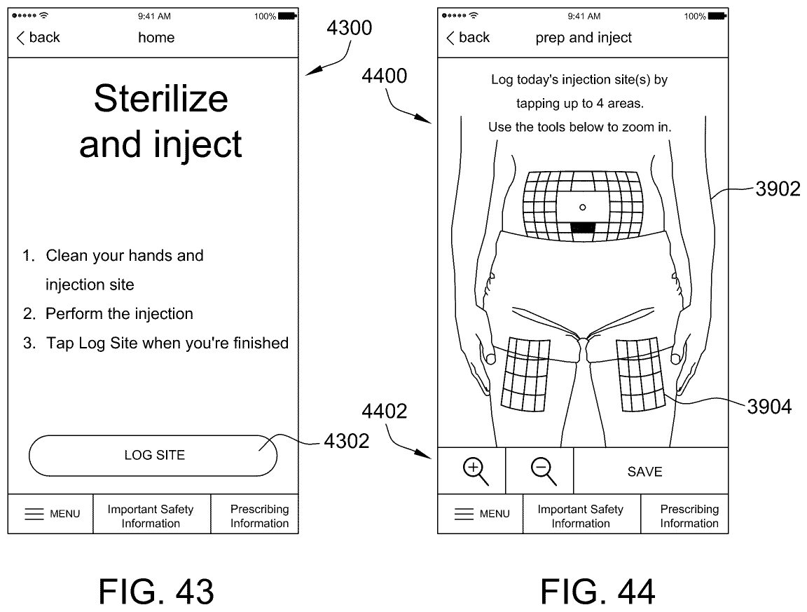

FIG. 40 illustrates a third injection preparation page 4000. The third injection preparation page 4000 includes a list 4002 of items that the patient should have prior to beginning their injection process. The list 4002 includes the device that the patient indicated (in their set-up process) is the device that they use to administer their medication. The patient may select a first option 4004 to view a tutorial on how to administer the medication (see FIG. 41), a second option 4006 to select a soundtrack to play during their injection (see FIG. 42), or a third option 4008 to begin the injection process without any additional aids (see FIG. 43).

FIG. 41 illustrates a video tutorial page 4100. The patient may view a tutorial video 4102 that shows the patient how they should administer their medication, based on the device (e.g., a pen or syringe) they have chosen to use to administer their medication. The patient may select the video 4102 to play, and may re-play the video 4102 as many times as necessary for the patient to feel sufficiently confident or prepared to perform the injection. Once the patient has watched the tutorial video 4102 and administered their injection, the patient may select an option 4104 to log the site of their injection directly after administering. The patient is also able to view the tutorial video 4102 before or after initiating injection preparation. In particular, the patient may view the tutorial video by selecting the “training” option 3504 on the home screen 3500, as shown in FIG. 35.

FIG. 42 illustrates a soundtrack selection page 4200. If the patient does not need a tutorial on how to administer their medication, the patient may use the soundtrack selection page 4200 to choose, in the example embodiment, an instrumental song to play during their injection. The songs available on the patient application are provided to calm, relax, and/focus the patient during their injection. Once the patient has administered their injection, the patient may select an option 4202 to log the site of their injection directly after administering.

FIG. 43 illustrates an injection administration page 4300. The injection administration page 4300 includes simple instructions for the patient to perform their injection without any additional aids (e.g., without a tutorial video or soundtrack). Once the patient has administered their injection, the patient may select an option 4302 to log the site of their injection directly after administering.

FIG. 44 illustrates a first injection logging page 4400. As shown above with respect to the last injection page 3900 in FIG. 39, a body image 3902 of a man or a woman is displayed with grid lines 3904 overlaid thereupon to indicate appropriate injection sites. Instructions guide the patient to tap areas that correspond to the sites on their own body at which they have administered their injection(s). The patient may tap any square defined by the grid lines 3904 to select the corresponding site, and the site will be activated (e.g., colored in or otherwise highlighted). In the example embodiment, the patient may tap an activated site again to de-activate the site. The injection logging page 4400 may include zoom tools 4402 for the patient to zoom into an area of interest. The patient may also drag the body image 3902, at any level of zoom, to view different areas on the body image 4902.

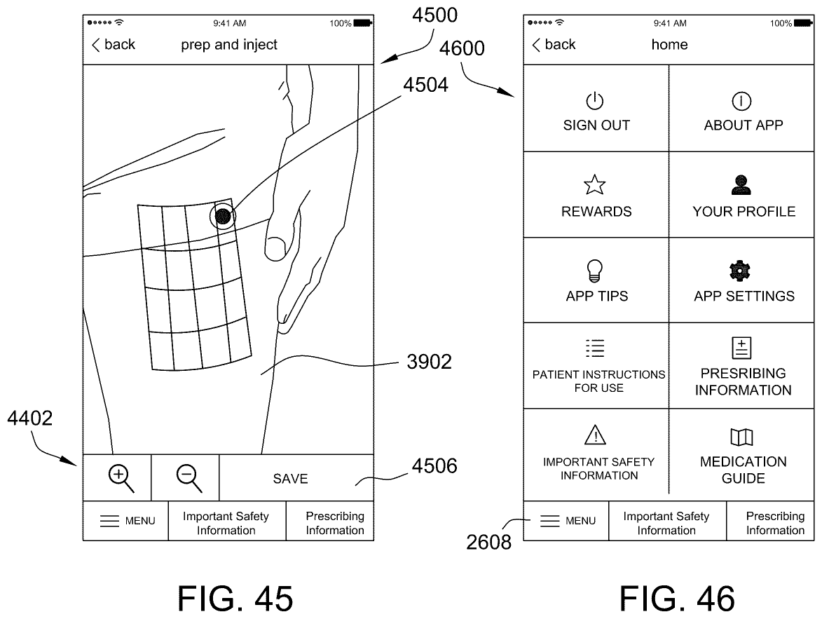

FIG. 45 illustrates a second, zoomed-in injection logging page 4500. In the example embodiment, the patient has used the zoom tools 4402 to zoom into a left leg of the body image 3902. Additionally, the patient has activated a site, as shown by the dark circle 4504 present in the active site. Up to four sites may be activated in a single logging session. The patient may select a “save” option 4506 to complete the injection logging process. After logging their injection site(s) and saving, the patient is returned to the home screen 3500 (shown in FIG. 35) of the patient application.

FIG. 46 illustrates the menu 4600 of the patient application, which is displayed upon selection of the menu option 2608, also shown in FIG. 26. The menu 4600 allows the patient to navigate several features of the patient application, including their user profile (e.g., the information provided during the registration and/or set-up phase, which they may update by selecting the “Profile” option in the menu), tips and/or FAQs on how to operate the patient application, application settings, and various information associated with the medication that may be useful for the patient to access and/or view. The menu 4600 may also include an option (not shown) for the patient to view their how their symptom(s) have been logged over time (e.g., to see progression such as a reduction of or improvement in symptoms) or how their progress or level of compliance compares to other similar users, based on their longitudinal profiles, as described herein. For example, the patient may select a “Progress” option to view a line graph or a bar graph showing the progression of the patient's symptoms and/or the patient's comparative level of treatment regimen compliance.

FIG. 47 illustrates an injection log page 4700 of the patient application. The injection log page 4700 may be displayed upon selection by the patient of the “injections” option 3508 on the home screen 3500, as shown in FIG. 35. An injection log 4702 is pre-populated to reflect logs 4704 from the current year. Logs 4704 from previous years may be available to view by swiping left or right. The “Variable Date” fields are populated to correspond to dates of injections logs 4704 created by the patient after injections. The patient may select one of the logs 4704 to view the associated date, time, and/or injection site(s), as illustrated on the body image 3902 shown in FIGS. 39 and 44-45. The logs 4704 include zoom tools 4706 for the patient to zoom in on areas of interest on the body image 3902. The patient may also edit a log 4704 by activating/deactivating a particular site, as described above. For any incomplete logs 4704 (e.g., the patient did not log any injection site(s) for a particular scheduled injection), the patient may update the log 4704 by logging sites or selecting a corresponding date/time, may initiate injection preparation if they have been directed by their HCP to take a missed injection, or may leave the log 4704 as incomplete (e.g., if their HCP has directed the patient not to inject for that scheduled injection). In one embodiment, the patient may be able to share one or more injection log(s) 4704 with another party (e.g., their HCP and/or medication ambassador) using a “share” command (not shown). The patient may be able to select the one or more injection log(s) 4704 they wish to share and the parties with whom they wish to share the log(s) 4704.

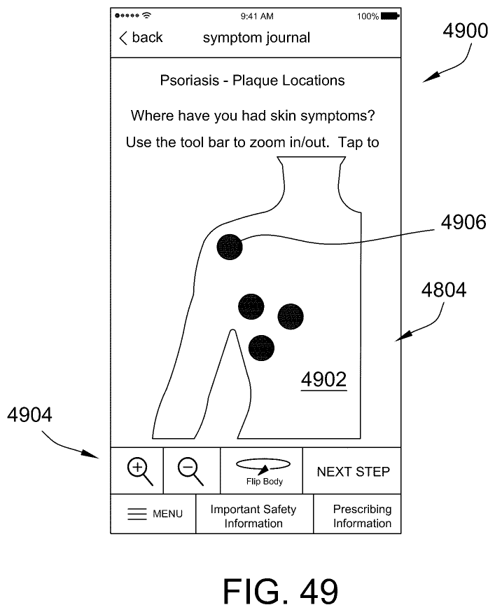

FIG. 48 illustrates a symptom journal page 4800 of the patient application. The symptom journal page 4800 may be displayed upon selection by the patient of the “symptom journal” option 3510 on the home screen 3500, as shown in FIG. 35. A symptom journal 4802 enables the patient to track the symptom(s) associated with their condition (input during the set-up phase). The patient may select an option 4806 to create a new symptom log 4804 (see FIG. 49) or may view previous symptom logs 4804. In the example embodiment, a predetermined number of symptom logs 4804 (e.g., one symptom log 4804) may be added per week. Accordingly, after the patient creates a symptom log 4804, the “log symptoms” option 4806 will be deactivated for a week. In other embodiments, more or fewer symptom logs 4804 may be added per week (or any other time interval). Additionally, in the example embodiment, the patient may not be able to edit or delete previous logs 4804.

FIG. 49 illustrates a new symptom log 4804. In the example embodiment, the patient is logging symptoms of their psoriasis. The symptom log 4804 provides a body silhouette 4902 that the patient may manipulate to indicate sites corresponding to their own symptoms. The body silhouette 1902 may be flipped (e.g., from front to back) and has zoom tools 4904 to zoom in and out on areas of interest. The patient may tap or otherwise select various sites on the body silhouette 4902 to activate sites 4906 that correspond to their own symptoms. Tapping an active or highlighted site 4906, as indicated by a darkened circle, de-activates the active site 4906. In the example embodiment, the patient has zoomed in on a shoulder region of the body silhouette 4902 to activate sites 4906 corresponding to their own symptoms. The patient may save the body silhouette 4902 including the active sites 4906 in the symptoms log 4804, and may have an option to add text or notes to the log with any additional information they would like to include in the log 4804 (such as, for example, internal, emotional, or physiological symptoms, or the number of times in a week or other interval that their symptoms have impacted their daily activities). The symptoms log 4804 may additionally or alternatively include various patient-reported outcomes (PRO) assessments, scales, or surveys that the patient may use to scale, rank, quantify, and/or characterize various symptoms and a severity or nature thereof (e.g., a level of pain experienced by the patient that day). In one embodiment, the patient may be able to share one or more symptom log(s) 4804 with another party (e.g., their HCP and/or medication ambassador) using a “share” command (not shown). The patient may be able to select the one or more symptom log(s) 4804 they wish to share and the parties with whom they wish to share the log(s) 4804.

In other embodiments, the patient application may include additional features and functionality. For example, the patient application may present a user interface to the patient including an option for patient to view or input additional data to their profile, including, for example, the results of lab tests or other clinical measures. The user interface may further include an option to access (e.g., import or view) data such as electronic medical records (e.g., health data, medication records, etc.) for the patient from another source, such as another application on the patient's computing device or from another connected device. The patient application may additionally provide an option for the patient to view their insurance information and how their insurance impacts their prescription. For example, the patient may be able to view a benefits verification and/or prior authorization timeline or status. In one embodiment, a benefits verification may be performed substantially instantaneously from within the patient application. Such information may be accessed (e.g., retrieved or imported) from another source, including a pharmacy data source (e.g., pharmacy data source 508), a benefits verifier (e.g., benefits verifier 602), the patient's insurance provider, and/or the patient's HCP, for example. The patient may use the patient application to check their eligibility for a medication copay savings card and, if eligible, may request to receive the copay savings card through the patient application.

The patient application may additionally enable prescription tracking and management through a user interface of the patient application. For example, the patient may use the patient application to order a prescription refill, set refill or delivery reminders, and/or view prescriber and/or pharmacy information. In some embodiments, the patient application activates a barcode scanner coupled to the computing device executing the patient application and scans a barcode on a received shipment of the medication. The patient application then transmits confirmation of receipt of the medication to a server computer, based on the scanned barcode. The patient application may further facilitate the ordering, tracking, and management of medication-related materials. For example, in some implementations, the patient application displays medication shipping data including a date that the medication was shipped, an expected delivery date, and a tracking number. This allows the patient to use the patient application to track shipments of the prescription product or additional products related to the prescription product. For example, the patient may use the patient application to order a sharps container for safe disposal of injection devices, and/or may manage injection training appointments through the patient application. Shipment tracking information displayed to the patient may be generated, for example, based on data in pharmacy data sources, such as those described herein.

The patient application may further provide one or more commands for the patient to share various elements of their profile and/or their injection and/or symptom logs. For example, the menu 4600 or home screen 3500 may include an option for the patient to share their application utilization data with another party, such as their HCP and/or their medication ambassador. The application utilization data may include when the patient has used the application, how often the patient uses the application, how often the patient tracks or logs their injections and/or their symptoms, the number of rewards the patient has earned, etc. Accordingly, the patient application may include HCP and/or medication ambassador contact information. For a patient using the patient application without a medication ambassador, the patient application may provide an option to contact and/or request a medication ambassador (and/or entry into a patient support program including a medication ambassador).

The patient application may further enable patients to set unique goals for themselves, for example exercise or productivity goals. The patient application may include options for the patient to edit a goal, to edit their progress towards a goal, and to set reminders or notifications regarding their progress towards a goal. In some implementations, the patient application displays alerts, reminders, and/or notifications for the patient to perform a task associated with one or more of the goals. Additionally, the patient application may provide rewards for setting goals and/or for completing goals. In some embodiments, as a further incentive, the patient application may provide access to a social platform, to enable communication with other users of the medication. The patient may ask questions or advice of other users, may share news about their personal progress (e.g., sharing rewards, completed goals, activities, progress), and may receive news and/or encouragement from other users.

In additional embodiments, a specialty pharmacy (or any other pharmacy data source 508, shown in FIG. 5) may employ a validated or specialized scale or metric to capture PROs and other clinical measures for those patients that do not use the patient application.

A computer-implemented method for generating patient adherence information for a patient taking a prescription product is provided. The method includes receiving, at a mobile computing device, medication schedule information for the prescription product, generating, based on the medication schedule information, a medication schedule, storing the medication schedule on the mobile computing device, generating, at the mobile computing device, a medication reminder based on the medication schedule, receiving, at the mobile computing device, in response to the medication reminder, patient input indicating whether the patient administered a dose of the prescription product, and generating, at the mobile computing device, patient adherence information based on the received patient input.

In one embodiment, the method for generating patient adherence information further includes transmitting the generated patient adherence information to a central data hub for comparison with patient adherence information associated with other patients.

In one embodiment, the method for generating patient adherence information further includes generating, using the mobile computing device, a missed-dose notification when the patient fails to administer the dose of the prescription product.

In one embodiment, generating a medication reminder includes generating a medication reminder that allows the patient to initiate a countdown timer for administration of the dose of the prescription product.

In one embodiment, generating a medication reminder includes generating a medication reminder that identifies a previous injection site associated with a previously administered dose of the prescription product.

In one embodiment, generating a medication reminder includes generating a medication reminder that includes a tutorial on how to administer the dose of the prescription product.

In one embodiment, the method for generating patient adherence information further includes receiving, at the mobile computing device, a user input placing an order for medication-related materials, initiating delivery of the medication-related materials based on the user input, and displaying, on the mobile computing device, shipping information associated with the delivery.

As will be appreciated based on the foregoing specification, the above-described embodiments of the disclosure may be implemented using computer programming or engineering techniques including computer software, firmware, hardware or any combination or subset thereof, wherein the technical effect of the systems and processes described herein is achieved by creating a network-based system for generating and analyzing longitudinal data profiles. Any such resulting program, having computer-readable code means, may be embodied or provided within one or more computer-readable media, thereby making a computer program product, e.g., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

This written description uses examples to disclose the embodiments, including the best mode, and also to enable any person skilled in the art to practice the disclosure, including making and using any devices or systems and performing any incorporated methods. The patentable scope of the embodiments is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer-implemented method for generating a longitudinal data profile from multiple disparate data sources, the method comprising:
storing, at a central data hub, first de-identified data received from a first data source, the first de-identified data including a plurality of data records having encrypted identifying data and an anonymous ID assigned to each record, wherein the anonymous ID is assigned based on a master list that includes a list of identifiers and corresponding anonymous IDs for each identifier;
storing, at the central data hub, second de-identified data received from a second data source, the second de-identified data including a plurality of data records having encrypted identifying data and an anonymous ID assigned to each record, wherein the anonymous ID is assigned based on the master list;
storing, at the central data hub, third de-identified data received from a third data source, the third de-identified data including a plurality of data records having encrypted identifying data and an anonymous ID assigned to each record, wherein the anonymous ID is assigned based on the master list;
processing, at the central data hub, the first, second, and third de-identified data to link the first, second, and third de-identified data using the anonymous ID; and
generating, at the central data hub, the longitudinal data profile from the linked first, second, and third de-identified data by organizing the first, second, and third de-identified data to generate a comprehensive profile that includes data from multiple disparate data sources.

2. The computer-implemented method of claim 1, further comprising:
receiving, at a data analyzer computing device communicatively coupled to the central data hub, one or more user inputs, wherein the one or more user inputs define a subset of longitudinal data profiles stored at the central data hub, and wherein the one or more user inputs identify an organizational scheme for the subset of longitudinal data profiles;
generating, at the central data hub, an extract based on the one or more user inputs, wherein the extract includes the subset of longitudinal data profiles with each longitudinal data profile in the subset organized according to the organizational scheme;
transmitting the extract from the central data hub to the data analyzer computing device; and
performing, at the data analyzer computing device, an analysis on the subset of longitudinal data profiles included in the extract.

3. A computer-implemented method for generating a longitudinal data profile from multiple disparate data sources, the method comprising:
storing, at a central data hub, first de-identified data received from a claims data source, the first de-identified data including a plurality of data records having encrypted identifying data and an anonymous ID assigned to each record, wherein the claims data source stores insurance claims data associated with medical or pharmaceutical insurance coverage for patients, wherein the insurance claims data is encrypted at the claims data source using an encryption algorithm, and wherein the anonymous ID is assigned based on a patient master list that includes a list of patient identifiers and corresponding anonymous IDs for each patient identifier;
storing, at the central data hub, second de-identified data received from a services data source, the second de-identified data including a plurality of data records having encrypted identifying data and an anonymous ID assigned to each record, wherein the services data source stores services data that indicates whether patients are enrolled in a patient support program, wherein the services data is encrypted using the encryption algorithm, and wherein the anonymous ID is assigned based on the patient master list;
storing, at the central data hub, third de-identified data received from a pharmacy data source, the third de-identified data including a plurality of data records having encrypted identifying data and an anonymous ID assigned to each record, wherein the pharmacy data source stores pharmacy data associated with a prescription product for patients, wherein the pharmacy data is encrypted using the encryption algorithm, and wherein the anonymous ID is assigned based on the patient master list;
processing, at the central data hub, the first, second, and third de-identified data to link the first, second, and third de-identified data using the anonymous ID; and
generating, at the central data hub, the longitudinal data profile for a patient from the linked first, second, and third de-identified data by organizing the first, second, and third de-identified data to generate a comprehensive history for the patient that includes data from multiple disparate healthcare data sources.

4. The computer-implemented method of claim 3, further comprising:
receiving, at a data analyzer computing device communicatively coupled to the central data hub, one or more user inputs, wherein the one or more user inputs define a subset of longitudinal data profiles stored at the central data hub, and wherein the one or more user inputs identify an organizational scheme for the subset of longitudinal data profiles;
generating, at the central data hub, an extract based on the one or more user inputs, wherein the extract includes the subset of longitudinal data profiles with each longitudinal data profile in the subset organized according to the organizational scheme;
transmitting the extract from the central data hub to the data analyzer computing device; and
performing, at the data analyzer computing device, an analysis on the subset of longitudinal data profiles included in the extract.

5. The computer-implemented method of claim 4, wherein performing an analysis comprises comparing a first plurality of longitudinal data profiles associated with patients who are enrolled in the patient support program against a second plurality of longitudinal data profiles associated with patients who are not enrolled in the patient support program to determine an efficacy of the patient support program.

6. The computer-implemented method of claim 5, wherein comparing the first plurality of longitudinal data profiles against the second plurality of longitudinal data profiles comprises comparing adherence data for the first and second plurality of longitudinal data profiles.

7. The computer-implemented method of claim 3, further comprising analyzing, using a data analyzer computing device communicatively coupled to the central data hub, the generated longitudinal data profile for at least one of health economics outcomes research and marketing analytics business intelligence research.

8. The computer-implemented method of claim 7, wherein analyzing the generated longitudinal data profile comprises generating an output that identifies a cost differential between a patient who participates in the patient support program and a patient who does not participate in the patent support program.

9. The computer-implemented method of claim 3, wherein the services data is encrypted using the encryption algorithm at a claims switch communicatively coupled to the services data source.

10. A central data hub for generating a longitudinal data profile from multiple disparate data sources, said central data hub configured to:
store first de-identified data received from a claims data source, the first de-identified data including a plurality of data records having encrypted identifying data and an anonymous ID assigned to each record, wherein the claims data source stores insurance claims data associated with medical or pharmaceutical insurance coverage for patients, wherein the insurance claims data is encrypted at the claims data source using an encryption algorithm, and wherein the anonymous ID is assigned based on a patient master list that includes a list of patient identifiers and corresponding anonymous IDs for each patient identifier;
store second de-identified data received from a services data source, the second de-identified data including a plurality of data records having encrypted identifying data and an anonymous ID assigned to each record, wherein the services data source stores services data that indicates whether patients are enrolled in a patient support program, wherein the services data is encrypted using the encryption algorithm, and wherein the anonymous ID is assigned based on the patient master list;
store third de-identified data received from a pharmacy data source, the third de-identified data including a plurality of data records having encrypted identifying data and an anonymous ID assigned to each record, wherein the pharmacy data source stores pharmacy data associated with a prescription product for patients, wherein the pharmacy data is encrypted using the encryption algorithm, and wherein the anonymous ID is assigned based on the patient master list;
process the first, second, and third de-identified data to link the first, second, and third de-identified data using the anonymous ID; and
generate the longitudinal data profile for a patient from the linked first, second, and third de-identified data by organizing the first, second, and third de-identified data to generate a comprehensive history for the patient that includes data from multiple disparate healthcare data sources.

11. The central data hub of claim 10, wherein said central data hub is further configured to:
receive, from a data analyzer computing device communicatively coupled to said central data hub, one or more user inputs, wherein the one or more user inputs define a subset of longitudinal data profiles stored at the central data hub, and wherein the one or more user inputs identify an organizational scheme for the subset of longitudinal data profiles; and
generate an extract based on the one or more user inputs, wherein the extract includes the subset of longitudinal data profiles with each longitudinal data profile in the subset organized according to the organizational scheme; and
transmit the extract to the data analyzer computing device.

12. The central data hub of claim 10, wherein the services data is encrypted using the encryption algorithm at a claims switch communicatively coupled to the services data source.

13. A computer-implemented method for directly providing a user with an estimated patient cost for a prescription product and a co-pay eligibility determination for the prescription product, the method comprising:
receiving, at a host computing device, from a remote user computing device, a user request for the estimated patient cost and the co-pay eligibility determination, wherein the user request includes patient identification information, and wherein the remote user computing device and the host computing device are linked together via a wide area network that includes the Internet;
retrieving, by the host computing device, from at least one database, benefits data and medication history data based on the patient identification information;
generating, at the host computing device, the estimated patient cost and the co-pay eligibility determination based on the patient identification information and the retrieved benefits data and medication history data; and
causing the estimated patient cost and the co-pay eligibility determination to be displayed on the remote user computing device.

14. The computer-implemented method of claim 13, wherein receiving a user request comprises receiving a user request from a prospective patient via a pre-check application running on the remote user computing device.

15. The computer-implemented method of claim 13, wherein receiving a user request comprises receiving a user request from a healthcare provider (HCP) via an HCP portal running on the remote user computing device.

16. The computer-implemented method of claim 13, further comprising:
retrieving electronic marketing data related to the prescription product;
analyzing, at the host computing device, the electronic marketing data to identify prospective patients;
storing the prospective patients in a prospective patient database;
comparing, at the host computing device, a patient database to the prospective patient database to identify patients; and
electronically evaluating, at the host computing device, the electronic marketing data based on the comparison.

17. The computer-implemented method of claim 13, wherein generating, at the host computing device, the estimated patient cost and the co-pay eligibility determination comprises generating the estimated patient cost based on a coverage status for the patient, an estimated cost for the prescription product, a deductible amount for the patient, an out of pocket limit for the patient, a minimum co-pay amount for the patient, a maximum co-pay amount for the patient, and a co-insurance amount for the patient.

18. The computer-implemented method of claim 13, wherein retrieving benefits data and medication history data comprises retrieving the benefits data and medication history data as structured XML data.

19. The computer-implemented method of claim 18, further comprising converting, at the computing device, the structured XML data into a PDF format.

20. The computer-implemented method of claim 19, wherein receiving a user request comprises receiving a user request that does not include prescription insurance information and prescription information.
